(19) United States
(12) Patent Application Publication
(10) Pub. No.: US 2004/0224969 A1
(43) Pub. Date: Nov. 11, 2004

(54) Salts of substituted 1,2,3,4-tetrahydroquinoline-2-carboxylic acid compounds

(75) Inventors: Corinna Sundermann, Aachen (DE); Michael Przewosny, Aachen (DE); Werner Guenter Englberger, Stolberg (DE)

Correspondence Address: CROWELL & MORING LLP, INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 10/770,123

(22) Filed: Feb. 3, 2004

Related U.S. Application Data
(63) Continuation of application No. PCT/EP02/08729, filed on Aug. 5, 2002.

(30) Foreign Application Priority Data
Aug. 3, 2001 (DE) 101 37 488.7

(51) Int. Cl. C07D 491/02; A61K 31/4741; A61K 31/473

(52) U.S. Cl. 514/290; 514/291; 546/79; 546/80

(57) ABSTRACT

Salts of substituted 1,2,3,4-tetrahydroquinoline-2-carboxylic acid compounds, processes for their preparation, drugs containing these compounds and their use in treatment methods and for the preparation of drugs for specific indications, especially for the treatment of pain.

### CROSS-REFERENCE TO RELATED APPLICATIONS

This application is a continuation of International Patent Application No. PCT/EP02/08729, filed Aug. 5, 2002, designating the United States of American, and published in German as WO 03/013530, the entire disclosure of which is incorporated herein by reference. Priority is claimed based on Federal Republic of Germany Patent Application No. DE 101 37 488.7, filed Aug. 3, 2001.

### FIELD OF THE INVENTION

The present invention relates to salts of substituted 1,2,3,4-tetrahydroquinoline-2-carboxylic acid derivatives, to processes for their preparation, to drugs containing these compounds and to their use for the preparation of drugs for specific indications, especially for the treatment of pain.

### BACKGROUND OF THE INVENTION

The treatment of chronic and non-chronic pain conditions is of great importance in medicine. There is a worldwide need for highly effective pain therapies for the patient-orientated and targeted treatment of chronic and non-chronic pain conditions, this being understood as meaning the successful and satisfactory treatment of pain for the patient. This manifests itself in the large number of scientific studies which have recently appeared in the field of applied analgesis or fundamental research into nociception.

Conventional opioids such as morphine are highly effective in the therapy of intense to very intense pain. However, their use is limited by the known side effects, e.g. respiratory depression, vomiting, sedation, constipation and the development of tolerance. Also, they are less effective in cases of neuropathic or incidental pain, as suffered by tumor patients in particular.

Opioids develop their analgesic effect by binding to membrane-based receptors belonging to the family of the so-called G protein coupled receptors. The biochemical and pharmacological characterization of subtypes of these receptors has now aroused the hope that subtype-specific opioids possess a different action/side effect profile from that of e.g. morphine. Further pharmacological studies have since suggested the probable existence of several subtypes of these opioid receptors (μ₁, μ₂, κ₁, κ₂, κ₃, δ₁ and δ₂).

In addition, there are other receptors and ion channels which play a substantial part in the system of the development and transmission of pain. The NMDA ion channel is of particular importance here because a substantial proportion of the communication of synapses passes through it. The calcium ion exchange between a neuronal cell and its environment is controlled by this channel.

Knowledge about the physiological significance of ion channel-selective substances has been gained by the development of the patch-clamp technique. The action of NMDA antagonists on the influx of calcium ions into the interior of the cell can be unambiguously demonstrated in this way. It has also been shown that these substances possess an independent antinociceptive potential (e.g. ketamine). It is important here that the mechanism of action is quite different than, e.g., in the case of opiates, because NMDA antagonists intervene directly in the decisive calcium balance of the cells during the transmission of pain. This affords the possibility for the first time of treating neuropathic forms of pain successfully.

Various NMDA antagonists, in this case involving tetrahydroquinoline derivatives, have already been described in the articles J. Med. Chem. (1992) 35, 1954-1968, J. Med. Chem. (1992) 35, 1942-1953 and Med. Chem. Res. (1991) 1, 64-73, and in patent applications EP 386 839, WO 97/12879 A1, WO 98/07704 A1 and WO 98/42673 A1. These references, especially the patent applications, provide a large number of possible indications, including pain therapy. However, the efficacy and applicability of these substances is still open to improvement, so there is a need for other substances here.

### SUMMARY OF THE INVENTION

One object of the invention was to provide analgesically effective substances, especially NMDA antagonists, which are suitable for pain therapy, including chronic and neuropathic pain in particular. Furthermore, these substances should exhibit a minimum of side effects, e.g. nausea, vomiting, dependence, respiratory depression or constipation.

Accordingly, the invention provides 1,2,3,4-tetrahydroquinoline-2-carboxylic acid derivatives of general formula I in the form shown, or in the form of their acids or their bases, or in the form of their salts, especially the physiologically acceptable salts, or in the form of their solvates, especially the hydrates, particularly in the form of their physiologically acceptable salts with cations or bases or with anions or acids, optionally in the form of their racemates or their pure stereoisomers, especially enantiomers or diastereoisomers, or in the form of mixtures of the stereoisomers, especially the enantiomers or diastereoisomers, in any desired mixing ratio:

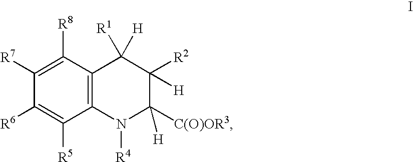

in which

either

R¹ and R² together form the following, each of which is monosubstituted or polysubstituted or unsubstituted:

—(CH₂)ₙ—, where n=3-10,

—CH═CH—CH₂—, —CH₂—CH═CH—,

—CH═CH—CH₂—CH₂—, —CH₂—CH₂—CH═CH—,

—CH₂—CH═CH—CH₂—,

—CH₂—CH═CH—CH₂—CH₂—, —CH₂—CH₂—CH═CH—CH₂—,

—CH₂—CH₂—CH═CH—CH₂—CH₂—,

—O—CH₂—CH₂—, —CH₂—CH₂—O—,

—O—CH₂—CH₂—CH₂—, —CH₂—CH₂—CH₂—O—,

—CH₂—O—CH₂—,

—CH₂—CH₂—O—CH₂—, —CH₂—O—CH₂—CH₂

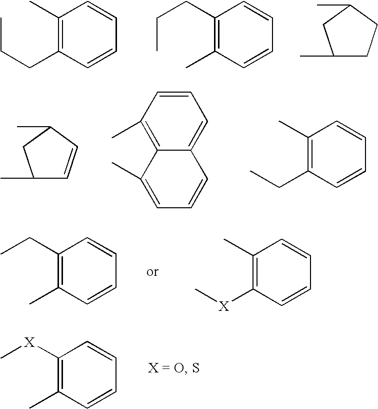

R³ is selected from

H; C₁-C₁₈-alkyl, C₂-C₁₈-alkenyl or C₂-C₁₈-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by N, S or O; alkylaryl or alkylheteroaryl, each of which is monosubstituted or polysubstituted or unsubstituted; and aryl or heteroaryl, each of which is monosubstituted or polysubstituted or unsubstituted;

R⁴ is selected from

R^{4a} or ZR^{4a}, where Z=C₁-C₆-alkyl, C₂-C₆-alkenyl or C₂-C₆-alkynyl, each of which is branched or unbranched and monosubstituted or polysubstituted or unsubstituted; and R^{4a} is selected from

H; C₁-C₁₂-alkyl, C₂-C₁₂-alkenyl or C₂-C₁₂-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; and aryl or heteroaryl, each of which is monosubstituted or polysubstituted or unsubstituted;

C(O)R⁹, C(O)OR⁹, C(S)R⁹, C(S)OR⁹ or S(O₂)R⁹, where R⁹ is selected from

H; C₁-C₁₀-alkyl, C₂-C₁₀-alkenyl or C₂-C₁₀-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; alkylaryl or alkylheteroaryl, each of which is monosubstituted or polysubstituted or unsubstituted; and aryl or heteroaryl, each of which is monosubstituted or polysubstituted or unsubstituted, especially phenethyl, 1-adamantyl, 2-adamantyl, 1-naphthyl or 2-naphthyl, 2-, 3- or 4-pyridyl or thiazolyl;

SR¹⁰, where R¹⁰ is selected from

aryl or heteroaryl, each of which is monosubstituted or polysubstituted or unsubstituted;

C(O)NR¹¹R¹², C(O)NR¹¹NR¹²R¹³, C(NR¹¹)NR¹²R¹³, C(S)NR¹¹R¹² or C(S)NR¹¹NR¹²R¹³, where R¹¹, R¹² and R¹³ independently of one another are selected from

H; C₁-C₁₈-alkyl, C₂-C₁₈-alkenyl or C₂-C₁₈-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; alkylaryl or alkylheteroaryl, each of which is monosubstituted or polysubstituted or unsubstituted; and aryl or heteroaryl, each of which is monosubstituted or polysubstituted or unsubstituted;

R⁵, R⁶, R⁷ and R⁸ independently of one another are selected from

H; F; Cl; Br; I; CN; NO₂; and C₁-C₁₀-alkyl, C₂-C₁₀-alkenyl or C₂-C₁₀-alkynyl, each of which is branched or unbranched and monosubstituted or polysubstituted or unsubstituted;

OR¹⁴, OC(O)R¹⁴, OC(S)R¹⁴, C(O)R¹⁴, C(O)OR¹⁴, C(S)R¹⁴, C(S)OR¹⁴, SR¹⁴, S(O)R¹⁴ or S(O₂)R¹⁴, where R¹⁴ is selected from

H; C₁-C₁₀-alkyl, C₂-C₁₀-alkenyl or C₂-C₁₀-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; alkylaryl or alkylheteroaryl, each of which is monosubstituted or polysubstituted or unsubstituted; and aryl or heteroaryl, each of which is monosubstituted or polysubstituted or unsubstituted;

NR¹⁵R¹⁶, NR¹⁵C(O)R¹⁶, C(NR¹⁵)NR¹⁶R¹⁷, NR¹⁵C(S)R¹⁶, C(S)NR¹⁵R¹⁶, C(S)NR¹⁵NR¹⁶R¹⁷ or S(O₂)NR¹⁵R¹⁶, where R¹⁵, R¹⁶ and R¹⁷ independently of one another are selected from

H; O; C₁-C₁₈-alkyl, C₂-C₁₅-alkenyl or C₂-C₁₈-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; alkylaryl or alkylheteroaryl, each of which is monosubstituted or polysubstituted or unsubstituted; and aryl or heteroaryl, each of which is monosubstituted or polysubstituted or unsubstituted; or

R¹⁵ and R¹⁶ or R¹⁶ and R¹⁷ together form a C₃-C₈-cycloalkyl which is saturated or unsaturated and monosubstituted or polysubstituted or unsubstituted, or a corresponding heterocycle in which at least one ring C atom is replaced by S, O or N; or

R⁵ and R⁶, R⁶ and R⁷ or R⁷ and R⁸ together form

═CR¹⁸—CH═CH—CH═ or ═CH—CR¹⁸═CH—CH═, where R¹⁸ is selected from H; F; Cl; Br; I; OH; and C₁-C₁₀-alkyl, C₂-C₁₀-alkenyl or C₂-C₁₀-alkynyl, each of which is branched or unbranched and monosubstituted or polysubstituted or unsubstituted,

with the proviso that

if R¹ and R² together form —CH═CH—CH₂— or

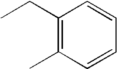

and R³ is (−)-p-menthan-3-ol, especially menthol or borneol, R⁷≠Cl and R⁵, R⁶ and R⁸≠H simultaneously,

if R¹ and R² together form —CH═CH—CH₂— and R³ is CH₃, R⁷: H, Cl or OCH₃ and R⁵, R⁶ and R⁸: H simultaneously,

if R^{1b} and R^{2a} together form —CH═CH—CH₂— and R³ is H, R⁷: OCH₃ or C(O)NH₂ and R⁵, R⁶ and R⁸: H, R⁵ and R⁷: CH₃ and R⁶ and R⁸: H, or R⁵≠OCH₃ and R⁶, R⁷ and R⁸: H simultaneously, or

if R^{1b} and R^{2a} together form

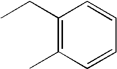

or —O—CH₂—CH₂— and R³ is C₂H₅, R⁷: H, Cl, CH₃, OCH₃ or NO₂ and R⁵, R⁶ and R⁸: H, or R⁵: NO₂ and R⁶, R⁷ and R⁸: H simultaneously; or

R¹ is selected from

C₁-C₁₀-alkyl which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; alkylaryl which is monosubstituted or polysubstituted or unsubstituted; and aryl which is monosubstituted or polysubstituted or unsubstituted;

OR¹⁹, SR¹⁹ or SO₂R¹⁹, where R¹⁹ is selected from

C₁-C₁₀-alkyl, C₂-C₁₀-alkenyl or C₂-C₁₀-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; and alkylaryl, aryl, alkylheteroaryl or heteroaryl, each of which is monosubstituted or polysubstituted or unsubstituted;

R² is selected from

H; C₁-C₁₀-alkyl which is branched or unbranched and monosubstituted or polysubstituted or unsubstituted; and phenyl which is monosubstituted or polysubstituted or unsubstituted, where if R² is phenyl, R¹ must be aryl, O-aryl or S-aryl;

R³ is selected from

H; C₁-C₁₈-alkyl, C₂-C₁₈-alkenyl or C₂-C₁₈-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by N, S or O;

alkylaryl or alkylheteroaryl, each of which is monosubstituted or polysubstituted or unsubstituted; and aryl or heteroaryl, each of which is monosubstituted or polysubstituted or unsubstituted;

R⁴ is selected from

R^{4a} or ZR^{4a}, where Z=C₁-C₆-alkyl, C₂-C₆-alkenyl or C₂-C₆-alkynyl, each of which is branched or unbranched and monosubstituted or polysubstituted or unsubstituted; and R^{4a} is selected from

H; C₁-C₁₂-alkyl, C₂-C₁₂-alkenyl or C₂-C₁₂-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; and aryl or heteroaryl, each of which is monosubstituted or polysubstituted or unsubstituted;

C(O)R⁹, C(O)OR⁹, C(S)R⁹, C(S)OR⁹ or S(O₂)R⁹, where R⁹ is selected from

H; C₁-C₁₀-alkyl, C₂-C₁₀-alkenyl or C₂-C₁₀-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; alkylaryl or alkylheteroaryl, each of which is monosubstituted or polysubstituted or unsubstituted; and aryl or heteroaryl, each of which is monosubstituted or polysubstituted or unsubstituted, especially phenethyl, 1-adamantyl, 2-adamantyl, 1-naphthyl or 2-naphthyl, 2-, 3- or 4-pyridyl or thiazolyl;

SR¹⁰, where R¹⁰ is selected from aryl or heteroaryl, each of which is monosubstituted or polysubstituted or unsubstituted;

C(O)NR¹¹R¹², C(O)NR¹¹NR¹²R¹³, C(NR¹¹)NR¹²R¹³, C(S)NR¹¹R¹² or C(S)NR¹¹NR¹²R¹³, where R¹¹, R¹² and R¹³ independently of one another are selected from

H; C₁-C₁₈-alkyl, C₂-C₁₈-alkenyl or C₂-C₁₈-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; alkylaryl or alkylheteroaryl, each of which is monosubstituted or polysubstituted or unsubstituted; and aryl or heteroaryl, each of which is monosubstituted or polysubstituted or unsubstituted;

R⁵, R⁶, R⁷ and R⁸ independently of one another are selected from

H; F; Cl; Br; I; CN; NO₂; and C₁-C₁₀-alkyl, C₂-C₁₀-alkenyl or C₂-C₁₀-alkynyl, each of which is branched or unbranched and monosubstituted or polysubstituted or unsubstituted;

OR¹⁴, OC(O)R¹⁴, OC(S)R¹⁴, C(O)R¹⁴, C(O)OR¹⁴, C(S)R¹⁴, C(S)OR¹⁴, SR¹⁴, S(O)R¹⁴ or S(O₂)R¹⁴, where R¹⁴ is selected from

H; C₁-C₁₀-alkyl, C₂-C₁₀-alkenyl or C₂-C₁₀-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; alkylaryl or alkylheteroaryl, each of which is monosubstituted or polysubstituted or unsubstituted; and aryl or heteroaryl, each of which is monosubstituted or polysubstituted or unsubstituted;

NR¹⁵R¹⁶, NR¹⁵C(O)R¹⁶, C(NR¹⁵)NR¹⁶R¹⁷, NR¹⁵C(S)R¹⁶, C(S)NR¹⁵R¹⁶, C(S)NR¹⁵NR¹⁶R¹⁷ or S(O₂)NR¹⁵R¹⁶, where R¹⁵, R¹⁶ and R¹⁷ independently of one another are selected from

H; O; C₁-C₁₈-alkyl, C₂-C₁₈-alkenyl or C₂-C₁₈-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; alkylaryl or alkylheteroaryl, each of which is monosubstituted or polysubstituted or unsubstituted; and aryl or heteroaryl, each of which is monosubstituted or polysubstituted or unsubstituted; or

R¹⁵ and R¹⁶ or R¹⁶ and R¹⁷ together form a C₃-C₈-cycloalkyl which is saturated or unsaturated and monosubstituted or polysubstituted or unsubstituted, or a corresponding heterocycle in which at least one C atom is replaced by S, O or N; or

R⁵ and R⁶, R⁶ and R⁷ or R⁷ and R⁸ together form

═CR¹⁸—CH═CH—CH═ or ═CH—CR¹⁸═CH—CH═, where R¹⁸ is selected from H; F; Cl; Br; I; OH; and C₁-C₁₀-alkyl, C₂-C₁₀-alkenyl or C₂-C₁₀-alkynyl, each of which is branched or unbranched and monosubstituted or polysubstituted or unsubstituted,

with the proviso that

if R⁴, R⁶, R⁷ and R⁸═H,

R¹≠CH₃, R³≠H or CH₃ and R² and R⁵≠H simultaneously; or

R¹ ≠unsubstituted phenyl, R³≠C₂H₅ and R² and R⁵ ≠H simultaneously;

if R⁴, R⁵, R⁶ and R⁸=H,

R¹≠S-phenyl, R²≠H, R⁷: Cl and R³≠CH₃ simultaneously; or

R¹≠S-2-pyridinyl, R²≠CH₃, R⁷: OCH₃ and R³: —CH₃—CH═CH₂ simultaneously; or

if R², R⁴, R⁵ and R⁷═H and R⁶ and R⁸=Cl,

R¹≠dioxalane and R³ ≠—CH₂—CH₂—OH simultaneously.

The 1,2,3,4-tetrahydroquinoline-2-carboxylic acid derivatives or their salts according to the invention exhibit a pronounced analgesic action and are also NMDA antagonists which selectively attack at the glycine binding site.

In terms of the present invention, alkyl or cycloalkyl radicals are understood as meaning saturated and unsaturated (but not aromatic), branched, unbranched and cyclic hydrocarbons which can be unsubstituted or monosubstituted or polysubstituted. C₁₋₂-alkyl is C1- or C2-alkyl, C₁₋₃-alkyl is C1-, C2- or C3-alkyl, C₁₋₄-alkyl is C1-, C2-, C3- or C4-alkyl, C₁₋₅-alkyl is C1-, C2-, C3-, C4- or C5-alkyl, C₁₋₆-alkyl is C1-, C2-, C3-, C4-, C5- or C6-alkyl, C₁₋₇-alkyl is C1-, C2-, C3-, C4-, C5-, C6- or C7-alkyl, C₁₋₈-alkyl is C1-, C2-, C3-, C4-, C5-, C6-, C7- or C8-alkyl, C₁₋₁₀-alkyl is C1-, C2-, C3-, C4-, C5-, C6-, C7-, C8-, C9- or C10-alkyl and C₁₋₁₈-alkyl is C1-, C2-, C3-, C4-, C5-, C6-, C7-, C8-, C9-, C10-, C11-, C12-, C13-, C14-, C15-, C16-, C17- or C18-alkyl. Also, C₃₋₄-cycloalkyl is C3- or C4-cycloalkyl, C₃₋₅-cycloalkyl is C3-, C4- or C5-cycloalkyl, C₃₋₆-cycloalkyl is C3-, C4-, C5- or C6-cycloalkyl, C₃₋₇-cycloalkyl is C3-, C4-, C5-, C6- or C7-cycloalkyl, C₃₋₈-cycloalkyl is C3-, C4-, C5-, C6-, C7- or C8-cycloalkyl, C₄₋₅-cycloalkyl is C4- or C5-cycloalkyl, C₄₋₆-cycloalkyl is C4-, C5- or C6-cycloalkyl, C₄₋₇-cycloalkyl is C4-, C5-, C6- or C7-cycloalkyl, C₅₋₆-cycloalkyl is C5- or C6-cycloalkyl and C₅₋₇-cycloalkyl is C5-, C6- or C7-cycloalkyl. ‘Cycloalkyl’ also embraces saturated cycloalkyls in which one or 2 carbon atoms are replaced by the heteroatom S, N or O. However, ‘cycloalkyl’ also includes especially monounsaturated or polyunsaturated and preferably monounsaturated cycloalkyls without a heteroatom in the ring as long as the cycloalkyl is not an aromatic system. The alkyl or cycloalkyl radicals are preferably methyl, ethyl, vinyl(ethenyl), propyl, allyl(2-propenyl), 1-propynyl, methylethyl, butyl, 1-methylpropyl, 2-methyl-propyl, 1,1-dimethylethyl, pentyl, 1,1-dimethylpropyl, 1,2-dimethylpropyl, 2,2-dimethylpropyl, hexyl, 1-methylpentyl, cyclopropyl, 2-methylcyclopropyl, cyclopropylmethyl, cyclobutyl, cyclopentyl, cyclopentylmethyl, cyclohexyl, cycloheptyl or cyclooctyl, but also adamantyl, CHF₂, CF₃ or CH₂OH, as well as pyrazolinone, oxopyrazolinone, 1,4-dioxane or dioxolane.

In the context of alkyl and cycloalkyl, ‘substituted’ in terms of the present invention is understood as meaning the substitution of a hydrogen radical by F, Cl, Br, I, NH₂, SH or OH, ‘polysubstituted’ radicals being understood as meaning that the substitution takes place both on different atoms and on the same atoms several times with the same or different substituents, for example three times on the same C atom, as in the case of CF₃, or at different sites, as in the case of —CH(OH)—CH═CH—CHCl₂. Particularly preferred substituents here are F, Cl and OH.

‘(CH₂)₃₋₆’ is understood as meaning —CH₂—CH₂—CH₂—, —CH₂—CH₂—CH₂—CH₂—, —CH₂—CH₂—CH₂—CH₂—CH₂— and —CH₂—CH₂—CH₂—CH₂—CH₂—CH₂—, ‘(CH₂)₁₋₄’ is understood as meaning —CH₂—, —CH₂—CH₂—, —CH₂—CH₂—CH₂— and —CH₂—CH₂—CH₂—CH₂—, etc.

Aryl radicals are understood as meaning ring systems having at least one aromatic ring, but without heteroatoms in only one of the rings. Examples are phenyl, naphthyl, fluoroanthenyl, fluorenyl, tetralinyl or indanyl radicals, especially 9H-fluorenyl or anthracenyl radicals, which can be unsubstituted or monosubstituted or polysubstituted.

Heteroaryl radicals are understood as meaning heterocyclic ring systems having at least one unsaturated ring which contain one or more heteroatoms from the group comprising nitrogen, oxygen and/or sulfur, and which can also be monosubstituted or polysubstituted. Examples of the heteroaryl group which may be mentioned are furan, benzofuran, thiophene, benzothiophene, pyrrole, pyridine, pyrimidine, pyrazine, quinoline, isoquinoline, phthalazine, benzo-1,2,5-thiadiazole, benzothiazole, indole, benzotriazole, benzodioxolane, benzodioxane, carbazole, indole and quinazoline.

In the context of aryl and heteroaryl, substituted is understood as meaning the substitution of the aryl or heteroaryl with R²², OR²², a halogen, preferably F and/or Cl, a CF₃, a CN, an NO₂, an NR²³R²⁴, a C₁₋₆-alkyl(saturated), a C₁₋₆-alkoxy, a C₃₋₈-cycloalkoxy, a C₃₋₈-cycloalkyl or a C₂₋₆-alkylene.

The radical R²² is H, a C₁₋₁₀-alkyl radical, preferably a C₁₋₆-alkyl radical, an aryl or heteroaryl radical or an aryl or heteroaryl radical bonded via a C₁₋₃-alkylene group, it being impossible for these aryl and heteroaryl radicals to themselves be substituted by aryl or heteroaryl radicals. The radicals R²³ and R²⁴, which may be identical or different, are H, a C₁₋₁₀-alkyl radical, preferably a C₁₋₆-alkyl radical, an aryl or heteroaryl radical or an aryl or heteroaryl radical bonded via a C₁₋₃-alkylene group, it being impossible for these aryl and heteroaryl radicals to themselves be substituted by aryl or heteroaryl radicals. Alternatively, the radicals R²³ and R²⁴ together are CH₂CH₂OCH₂CH₂, CH₂CH₂NR²⁵CH₂CH₂ or (CH₂)₃₋₆, and the radical R²⁵ is H, a C₁₋₁₀-alkyl radical, preferably a C₁₋₆-alkyl radical, an aryl or heteroaryl radical or an aryl or heteroaryl radical bonded via a C₁₋₃-alkylene group, it being impossible for these aryl and heteroaryl radicals to themselves be substituted by aryl or heteroaryl radicals.

‘Salt’ is understood as meaning any form of the active ingredient according to the invention in which it takes on an ionic (here usually anionic) form, or is charged, and is coupled with a counterion (here usually a cation) or is in solution. This is understood as including complexes of the active ingredient with other molecules and ions, especially complexes that are complexed via ionic interactions.

In terms of the present invention, ‘physiologically acceptable salts with cations or bases’ are understood as meaning salts of at least one of the compounds according to the invention—usually a (deprotonated) acid—as an anion with at least one, preferably inorganic cation, which are physiologically acceptable, especially when used in humans and/or mammals. Particularly preferred salts are those of alkali metals and alkaline earth metals and also NH₄+, but very particularly preferred salts are monosodium or disodium, monopotassium or dipotassium, magnesium or calcium salts.

In terms of the present invention, ‘physiologically acceptable salts with anions or acids’ are understood as meaning salts of at least one of the compounds according to the invention—usually protonated, e.g. on the nitrogen—as a cation with at least one anion, which are physiologically acceptable, especially when used in humans and/or mammals. In terms of the present invention, this is understood in particular as meaning the salts formed with physiologically acceptable acids, i.e. salts of the active ingredient in question with inorganic or organic acids, which are physiologically acceptable, especially when used in humans and/or mammals. Examples of physiologically acceptable salts of specific acids are salts of hydrochloric acid, hydrobromic acid, sulfuric acid, methanesulfonic acid, formic acid, acetic acid, oxalic acid, succinic acid, tartaric acid, mandelic acid, fumaric acid, lactic acid, citric acid, glutamic acid, 1,1-dioxo-1,2-dihydro1b6-benzo [d]isothiazol-3-one (saccharinic acid), monomethylsebacic acid, 5-oxoproline, hexane-1-sulfonic acid, nicotinic acid, 2-, 3- or 4-aminobenzoic acid, 2,4,6-trimethylbenzoic acid, α-lipoic acid, acetylglycine, acetylsalicylic acid, hippuric acid and/or aspartic acid. The hydrochloride salt is particularly preferred.

In terms of the present invention, preferred derivatives are the substituted 1,2,3,4-tetrahydroquinoline-2-carboxylic acid derivatives of formula I in the form of their physiologically acceptable salts with cations or bases. These salts are called ‘salts according to the invention’ or ‘salts according to the invention of substituted 1,2,3,4-tetrahydroquinoline-2-carboxylic acid derivatives of formula I’ in the description which follows. However, ‘salts according to the invention’ or ‘salts according to the invention of substituted 1,2,3,4-tetrahydro-quinoline-2-carboxylic acid derivatives of formula I’ are not necessarily restricted to physiologically acceptable salts of the substituted 1,2,3,4-tetrahydroquinoline-2-carboxylic acid derivatives of formula I with cations or bases, but can optionally also include selected free bases or free acids or physiologically acceptable salts with anions or acids.

One particularly preferred subject of the present invention consists of salts according to the invention of substituted 1,2,3,4-tetrahydroquinoline-2-carboxylic acid derivatives of formula I in which R⁴ is selected from

H; C₁-C₁₀-alkyl, C₂-C₁₀-alkenyl or C₂-C₁₀-alkynyl, each of which is branched or unbranched and monosubstituted or polysubstituted or unsubstituted; and C3-C₈-cycloalkyl which is saturated or unsaturated and monosubstituted or polysubstituted or unsubstituted; and

C(O)R⁹, where R⁹ is selected from

H; C₁-C₁₀-alkyl, C₂-C₁₀-alkenyl or C₂-C₁₀-alkynyl, each of which is branched or unbranched and monosubstituted or polysubstituted or unsubstituted; C₃-C₈-cycloalkyl which is saturated or unsaturated and monosubstituted or polysubstituted or unsubstituted; and aryl or heteroaryl, each of which is monosubstituted or polysubstituted or unsubstituted, especially phenethyl, 1-adamantyl, 2-adamantyl, 1-naphthyl or 2-naphthyl, 2-, 3- or 4-pyridyl or thiazolyl.

Particularly preferred salts according to the invention of substituted 1,2,3,4-tetrahydroquinoline-2-carboxylic acid derivatives of formula I are those in which R⁴ is selected from

H; C₁-C₁₀-alkyl which is unsubstituted or monosubstituted or polysubstituted; and phenyl which is unsubstituted or monosubstituted or polysubstituted, preferably H, CH₃ or C₂H₅ and especially H.

One preferred subject of the present invention consists of salts according to the invention of substituted 1,2,3,4-tetrahydroquinoline-2-carboxylic acid derivatives of formula I in which R³ is selected from

H; C₁-C₁₀-alkyl, C₂-C₁₀-alkenyl or C₂-C₁₀-alkynyl, each of which is branched or unbranched and monosubstituted or polysubstituted or unsubstituted; C3-C₈-cycloalkyl which is saturated or unsaturated and monosubstituted or polysubstituted or unsubstituted, or a corresponding heterocycle in which at least one ring C atom is replaced by N or O;

alkylaryl which is monosubstituted or polysubstituted or unsubstituted;

and aryl or heteroaryl, each of which is monosubstituted or polysubstituted or unsubstituted.

Particularly preferred salts according to the invention of substituted 1,2,3,4-tetrahydroquinoline-2-carboxylic acid derivatives of formula I are those in which R³ is selected from

H; C₁-C₄-alkyl which is branched or unbranched and monosubstituted or polysubstituted or unsubstituted; and phenyl, benzyl or phenethyl which is monosubstituted or polysubstituted or unsubstituted, preferably H, CH₃ or C₂H₅ and especially H.

Particularly preferred salts according to the invention of substituted 1,2,3,4-tetrahydroquinoline-2-carboxylic acid derivatives of formula I are those in which R¹ and R² together form —O—CH₂—CH₂—, (—CH₂—)ₙ where n=3-6, preferably 3 or 6, —CH═CH—CH₂—, —CH═CH—CH₂—CH₂—,

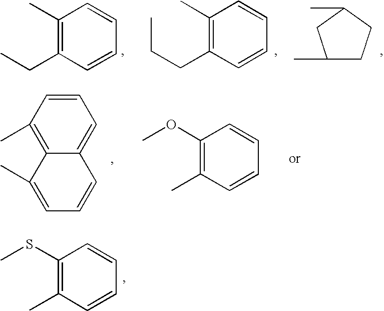

preferably —CH═CH—CH₂— or —CH═CH—CH₂—CH₂— and especially —CH═CH—CH₂—.

Another preferred subject of the invention consists of salts according to the invention of substituted 1,2,3,4-tetraquinoline-2-carboxylic acid derivatives of formula I in which R¹ is selected from

phenyl, naphthyl or anthracenyl which is unsubstituted or monosubstituted or polysubstituted; and OR¹⁹ or SR¹⁹, where R¹⁹ is selected from

C₁-C₆-alkyl which is branched or unbranched and monosubstituted or polysubstituted or unsubstituted; C₃-C₈-cycloalkyl which is saturated or unsaturated and monosubstituted or polysubstituted or unsubstituted; and aryl which is monosubstituted or polysubstituted or unsubstituted;

preferably anthracenyl, naphthyl or, in particular, phenyl which is unsubstituted or monosubstituted or polysubstituted by a substituent selected from

F; Cl; Br; I; methoxy; ethoxy; propoxy; methyl; ethyl; propyl(n-propyl, i-propyl); butyl(n-butyl, i-butyl, t-butyl); carboxyl; nitro; benzyloxy; phenyl; hydroxyl; phenoxy; trifluoromethyl; dioxolyl and SCH₃;

or OR¹⁹ or SR¹⁹, where R¹⁹ is selected from

C₁-C₄-alkyl which is branched or unbranched and monosubstituted or polysubstituted or unsubstituted; C₃-C₈-cycloalkyl which is saturated or unsaturated and monosubstituted or polysubstituted or unsubstituted; and aryl which is monosubstituted or polysubstituted or unsubstituted;

especially unsubstituted phenyl, naphthyl and anthracenyl, O-hydroxyethyl, ethoxynaphthyl, 4-hydroxy-3-methoxyphenyl, 4-propoxyphenyl, 2,3,4-trimethylphenyl, 2,4,5-trimethoxyphenyl, SCH₃, 2-chlorophenyl, 3-chlorophenyl, 4-chlorophenyl, 2-fluorophenyl, 3-fluorophenyl, 4-fluorophenyl, 2-bromophenyl, 3-bromophenyl, 4-bromophenyl, 2,6-dichlorophenyl, 4-carboxyphenyl, 3-nitrophenyl, 2,4,6-trimethylphenyl, 2,5-dimethylphenyl, 3,4-dimethoxyphenyl, 4-benzyloxy-3-methoxyphenyl, 3-methylphenyl, 4-methoxyphenyl, 4-biphenyl, 4-methylphenyl, 4-ethoxyphenyl, 2-methylphenyl, 2,4-dimethylphenyl, 2,6-dimethylphenyl, 4-hydroxy-3-methoxyphenyl, 4-methylhydroxyphenyl, 4-hydroxyphenyl, 4-phenoxyphenyl, 4-nitrophenyl, 4-chloromethylphenyl, 4-tert-butylphenyl, 3,5-bis(trifluoromethyl)phenyl, 4-acetoxyphenyl, 4-cyanophenyl, 2-methoxyphenyl, 2,6-difluorophenyl, 2-trifluoromethylphenyl, 3-trifluoromethylphenyl, 4-trifluoromethylphenyl, 3-methoxyphenyl, 2-, 3- or 4-benzyloxyphenyl, S-phenyl or 6-chlorobenzo[1,3]dioxol-5-yl.

Another preferred subject of the present invention consists of salts according to the invention of substituted 1,2,3,4-tetrahydroquinoline-2-carboxylic acid derivatives of formula I in which R² is selected from

H; C₁-C₄-alkyl which is branched or unbranched and monosubstituted or polysubstituted or unsubstituted; and phenyl which is monosubstituted or polysubstituted or unsubstituted, preferably H, unsubstituted phenyl, 4-methoxyphenyl or CH₃ and especially H.

One preferred subject of the present invention consists of salts according to the invention of substituted 1,2,3,4-tetrahydroquinoline-2-carboxylic acid derivatives of formula I in which R⁵, R⁶, R⁷ and R⁸ independently of one another are selected from

H; F; Cl; Br; I; CN; NO₂; and C₁-C₁₀-alkyl, C₂-C₁₀-alkenyl or C₂-C₁₀-alkynyl, each of which is branched or unbranched and monosubstituted or polysubstituted or unsubstituted;

OR¹⁴, C(O)R¹⁴, C(O)OR¹⁴ or SR¹⁴, R¹⁴ being selected from

H; C₁-C₁₀-alkyl, C₂-C₁₀-alkenyl or C₂-C₁₀-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; alkylaryl or alkylheteroaryl, each of which is monosubstituted or polysubstituted or unsubstituted; and aryl or heteroaryl, each of which is monosubstituted or polysubstituted or unsubstituted; and

NR¹⁵R¹⁶ or NR¹⁵C(O)R¹⁶, R¹⁵ and R¹⁶ independently of one another being selected from

H; O; C₁-C₁₀-alkyl, C₂-C₁₀-alkenyl or C₂-C₁₀-alkynyl, each of which is branched or unbranched and monosubstituted or polysubstituted or unsubstituted.

Particularly preferred salts according to the invention of substituted 1,2,3,4-tetrahydroquinoline-2-carboxylic acid derivatives of formula I are those in which R⁵, R⁶, R⁷ and R⁸ independently of one another are selected from

H; F; Cl; Br; I; CN; NO₂; and C₁-C₆-alkyl, C₂-C₆-alkenyl or C₂-C₆-alkynyl, each of which is branched or unbranched and monosubstituted or polysubstituted or unsubstituted;

OR¹⁴, C(O)R¹⁴, C(O)OR¹⁴ or SR¹⁴, R¹⁴ being selected from

H; C₁-C₄-alkyl which is branched or unbranched and monosubstituted or polysubstituted or unsubstituted; and aryl which is monosubstituted or polysubstituted or unsubstituted;

preferably, R⁵, R⁶, R⁷ and R⁸ independently of one another are selected from

H; F; Cl; Br; I; CN; and C₁-C₄-alkyl which is branched or unbranched and monosubstituted or polysubstituted or unsubstituted;

OR¹⁴ or SR¹⁴, R¹⁴ being selected from

C₁-C₄-alkyl which is branched or unbranched and monosubstituted or polysubstituted or unsubstituted; and aryl which is monosubstituted or polysubstituted or unsubstituted;

in particular, R⁵, R⁶, R⁷ and R⁸ independently of one another are selected from H; F; Cl; Br; I; CN; CH₃; CF₃; t-butyl; i-butyl; —OCH₃; —OCF₃; —SCH₃ and —O-phenyl.

Very particularly preferred salts according to the invention of substituted 1,2,3,4-tetrahydroquinoline-2-carboxylic acid derivatives of formula I are those in which

R⁵, R⁶ and R⁸ are H and R⁷ is Cl, or

R⁵ and R⁷ are H and R⁶ and R⁸ are Cl.

Particularly preferred subjects consist of the salts according to the invention of the following substituted 1,2,3,4-tetrahydroquinoline-2-carboxylic acid derivatives:

7,9-dichloro-3a,4,5,9b-tetrahydro-3H-cyclopenta [c]quinoline-4-carboxylic acid,

8-chloro-3a,4,5,9b-tetrahydro-3H-cyclopenta [c]quinoline-4-carboxylic acid,

6-chloro-7-trifluoromethyl-4-(2,4,6-trimethylphenyl)-1,2,3,4-tetrahydroquinoline-2-carboxylic acid,

4-(2-hydroxyethoxy)-6-trifluoromethoxy-1,2,3,4-tetrahydroquinoline-2-carboxylic acid,

6-iodo-4-(4-methoxyphenyl)-3-methyl-1,2,3,4-tetrahydroquinoline-2-carboxylic acid,

5,7-dichloro-4-phenyl-1,2,3,4-tetrahydroquinoline-2-carboxylic acid,

5,7-dichloro-4-m-tolyl-1,2,3,4-tetrahydroquinoline-2-carboxylic acid,

5,7-dichloro-4-p-tolyl-1,2,3,4-tetrahydroquinoline-2-carboxylic acid,

5,7-dichloro-4-(2,4-dimethylphenyl)-1,2,3,4-tetrahydroquinoline-2-carboxylic acid,

5,7-dichloro-4-(2-fluorophenyl)-1,2,3,4-tetrahydroquinoline-2-carboxylic acid,

5,7-dichloro-4-(3-fluorophenyl)-1,2,3,4-tetrahydroquinoline-2-carboxylic acid,

5,7-dichloro-4-(4-fluorophenyl)-1,2,3,4-tetrahydroquinoline-2-carboxylic acid,

5,7-dichloro-4-(2-chlorophenyl)-1,2,3,4-tetrahydroquinoline-2-carboxylic acid,

5,7-dichloro-4-(4-chlorophenyl)-1,2,3,4-tetrahydroquinoline-2-carboxylic acid,

5,7-dichloro-4-(3-bromophenyl)-1,2,3,4-tetrahydroquinoline-2-carboxylic acid,

5,7-dichloro-4-(4-bromophenyl)-1,2,3,4-tetrahydroquinoline-2-carboxylic acid,

7,8-dichloro-4-(2-chlorophenyl)-1,2,3,4-tetrahydroquinoline-2-carboxylic acid,

6-cyano-4-(2,3,4-trimethoxyphenyl)-1,2,3,4-tetrahydroquinoline-2-carboxylic acid,

6,8,9-trichloro-2,3,3a,4,5,9b-hexahydrofuro [3,2-c]quinoline-4-carboxylic acid,

8-methoxy-4-(4-methoxyphenyl)-3-methyl-1,2,3,4-tetrahydroquinoline-2-carboxylic acid,

5,6,8-trichloro-4-(4-hydroxyphenyl)-3-methyl-1,2,3,4-tetrahydroquinoline-2-carboxylic acid,

4-(3,4-dimethoxyphenyl)-8-iodo-1,2,3,4-tetrahydroquinoline-2-carboxylic acid,

6-iodo-4-(4-methylsulfanylphenyl)-1,2,3,4-tetrahydroquinoline-2-carboxylic acid,

4-(4-ethoxy-3-methoxyphenyl)-6-phenoxy-1,2,3,4-tetrahydroquinoline-2-carboxylic acid,

4-(2-ethoxynaphthalen-1-yl)-6-iodo-1,2,3,4-tetrahydroquinoline-2-carboxylic acid,

8-chloro-4-(4-propoxyphenyl)-1,2,3,4-tetrahydroquinoline-2-carboxylic acid,

4-(2,4-dimethoxy-3-methylphenyl)-6-phenoxy-1,2,3,4-tetrahydroquinoline-2-carboxylic acid,

4-anthracen-9-yl-6-chloro-8-methyl-1,2,3,4-tetrahydroquinoline-2-carboxylic acid,

6-sec-butyl-4-naphthalen-1-yl-1,2,3,4-tetrahydroquinoline-2-carboxylic acid,

4-(4-hydroxyphenyl)-3-methyl-8-phenoxy-1,2,3,4-tetrahydroquinoline-2-carboxylic acid,

8-chloro-6-fluoro-4-naphthalen-2-yl-1,2,3,4-tetrahydroquinoline-2-carboxylic acid,

4-(4-methoxyphenyl)-3-methyl-6-phenoxy-1,2,3,4-tetrahydroquinoline-2-carboxylic acid,

6-chloro-8-fluoro-4-m-tolyl-1,2,3,4-tetrahydroquinoline-2-carboxylic acid,

8-chloro-6-fluoro-4-m-tolyl-1,2,3,4-tetrahydroquinoline-2-carboxylic acid,

4-(4-bromophenyl)-6-chloro-8-fluoro-1,2,3,4-tetrahydroquinoline-2-carboxylic acid,

7,8-dichloro-4-(2,4-dimethylphenyl)-1,2,3,4-tetrahydroquinoline-2-carboxylic acid,

6-chloro-4-(4-chlorophenyl)-7-trifluoromethyl-1,2,3,4-tetrahydroquinoline-2-carboxylic acid,

4-(2-chlorophenyl)-6-cyano-1,2,3,4-tetrahydroquinoline-2-carboxylic acid,

6-bromo-8-chloro-4-(2,4-dimethylphenyl)-1,2,3,4-tetrahydroquinoline-2-carboxylic acid,

6-bromo-4-(2-bromophenyl)-8-chloro-1,2,3,4-tetrahydroquinoline-2-carboxylic acid,

4-(4-hydroxy-3-methoxyphenyl)-3-methyl-6-methylsulfanyl-1,2,3,4-tetrahydroquinoline-2-carboxylic acid,

6-cyano-3,4-bis(4-methoxyphenyl)-1,2,3,4-tetrahydroquinoline-2-carboxylic acid,

5,7-dichloro-4-(4-chlorophenyl)-1,2,3,4-tetrahydroquinoline-2-carboxylic acid,

5,7-dichloro-4-(3-chlorophenyl)-1,2,3,4-tetrahydroquinoline-2-carboxylic acid,

5,7-dichloro-4-(4-chlorophenyl)-1,2,3,4-tetrahydroquinoline-2-carboxylic acid,

1,3-dichloro-5,6,6a,7,8,12b-hexahydrobenzo [k]phenanthridine-6-carboxylic acid,

1,3-dichloro-5,6a,7,11b-tetrahydro-6H-indeno [2,1-c]quinoline-6-carboxylic acid,

5,7-dichloro-4-(3,5-dimethylphenyl)-1,2,3,4-tetrahydroquinoline-2-carboxylic acid and

7,9-dichloro-2,3,3a,4,5,9b-hexahydro-1H-cyclopenta [c]quinoline-4-carboxylic acid,

particular preference being given to sodium 7,9-dichloro-3a,4,5,9b-tetrahydro-3H-cyclopenta[c]quinoline-4-carboxylate or sodium 7,9-dichloro-2,3,3a,4,5,9b-hexahydro-1H-cyclopenta [c]quinoline-4-carboxylate, especially sodium 7,9-dichloro-3a,4,5,9b-tetrahydro-3H-cyclopenta [c]-quinoline-4-carboxylate.

Particularly preferred salts according to the invention of substituted 1,2,3,4-tetrahydroquinoline-2-carboxylic acid derivatives of formula I are the alkali metal salts, preferably the sodium or potassium salts and especially the sodium salts.

The invention also provides processes for the preparation of salts according to the invention of a substituted 1,2,3,4-tetrahydroquinoline-2-carboxylic acid derivative.

Various processes for the preparation of tetrahydroquinolines are described in the literature:

a solid phase preparation (WO 98/34111),

multistage procedures (WO 98/42673; Bioorganic and Medicinal Chemistry Letters, vol. 2, p. 371, 1992; Journal of Heterocyclic Chemistry, vol. 25, p. 1831, 1988; Journal of the Chemical Society, Perkin Transactions I (1989), page 2245) or

a Lewis acid-catalyzed “one-pot” process (Journal of the Chemical Society, Chemical Communications, 1999, p. 651; Journal of the American Chemical Society, vol. 118, p. 8977, 1996).

However, all these processes clearly have some disadvantages.

In contrast to these processes, the so-called basic process described here is a process mediated by trifluoroacetic acid—preferably a “one-pot” process—in which one aromatic amine component, one aldehyde component and one electron-rich olefin component are reacted with one another.

In the basic process, substituted 1,2,3,4-tetrahydroquinoline-2-carboxylic acid derivatives of formula I in which R⁴=H, the other radicals having one of the meanings already mentioned, are prepared first. Anilines of formula II, in which R⁵, R⁶, R⁷ and R⁸ each independently of one another have one of the meanings already indicated or are provided with a protecting group,

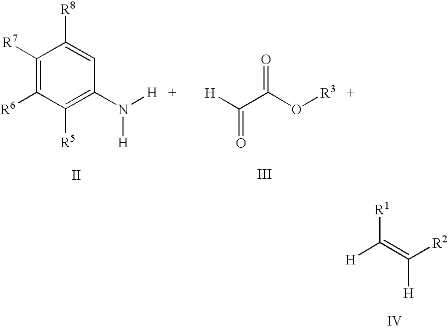

glyoxalic acid esters or optionally glyoxalic acid of formula III and olefins of formula IV, in which R¹, R² and R³ each independently of one another have one of the meanings already indicated or are provided with a protecting group, are reacted with trifluoroacetic acid at between 0° C. and 100° C. The reaction time is preferably 0.25-12 h and particularly preferably at most 2 h, the reaction is carried out preferably at a temperature of between 20° C. and 40° C. and particularly preferably at room temperature, and/or the reaction is a one-pot reaction. When this basic process has ended, any existing ester groups can be saponified and/or the product formed in the basic process can optionally be brought into contact with a strong base, which may already contain the desired cation, in order to form a salt.

One decisive advantage of the process according to the invention is that it produces the desired systems according to a domino reaction (imine formation and subsequent aza Diels-Alder reaction) with very high selectivities and with good yields.

Without having to carry out a linking or cleaving step as in the case of the solid phase preparation, and also without purification of the intermediates as in the case of the solution chemistry described, the process according to the invention is distinguished not only by its ease of implementation but also by its purification method. Washing several times with non-polar solvents, for example n-hexane, makes it possible for the most part to obtain the products in high purity. In other cases, they can be purified by means of column chromatography. In particular, the compounds of formula I can be obtained as the pure diastereoisomers by the washing processes with non-polar solvents, for example n-hexane, or by crystallization of their salts.

In general, in one advantageous embodiment of the preparative process, when the formation of a compound of formula I has ended, the compound is brought into contact with a strong base, which may already contain the desired cation, and the resulting salt according to the invention is then purified.

The majority of the reactants used here, especially those of formulae II, III and IV, are commercially available or can be prepared by simple synthesis steps known to those skilled in the art.

In consecutive reactions following the basic process, the products formed in the basic process can be converted to consecutive products of formula I according to the invention by a procedure known to those skilled in the art, whereby initially the hydrogen on R⁴ is substituted.

Thus, when the basic reaction has ended, if the product is to be a substituted 1,2,3,4-tetrahydroquinoline-2-carboxylic acid derivative of formula I where R⁴=alkylformyl, acyl, sulfenyl or sulfonyl, the reaction product can be reacted with the appropriate chloroformate or fluoroformate, acid chloride, sulfenyl chloride or sulfonyl chloride in the presence of a base, preferably triethylamine, pyridine or NaOH, in water, a dioxane/water mixture or a THF/water mixture, at a temperature of between 0 and 20° C. (J. Org. Chem., 1989, 54, 5574-5580).

Likewise, when the basic reaction has ended, if the product is to be a substituted 1,2,3,4-tetrahydroquinoline-2-carboxylic acid derivative of formula I where R⁴=C(S)NR¹¹R¹², the reaction product can be reacted with a thionating reagent, preferably Lawesson's reagent (2,4-bis(4-methoxyphenyl)-2,4-dithioxo-1,3,2,4-dithiaphosphetane), in an organic solvent, preferably THF or toluene, at a temperature of 30-50° C.

Alternatively, when the basic reaction has ended, if the product is to be a substituted 1,2,3,4-tetrahydroquinoline-2-carboxylic acid derivative of formula I where R⁴=C(O)NR¹¹R¹² or C(S)NR¹¹R¹², the reaction product can be reacted with potassium cyanate or potassium isothiocyanate in water at temperatures of up to 100° C., or with an organic isocyanate or isothiocyanate in an alcohol, preferably methanol, ethanol or isopropanol, at temperatures up to the boiling point.

Again, when the basic reaction has ended, if the product is to be a substituted 1,2,3,4-tetrahydroquinoline-2-carboxylic acid derivative of formula I where R⁴=C(NR¹¹)NR¹²R¹³, the reaction product can be reacted under alkaline conditions with an O-methylisourea or S-methylisothiourea at temperatures of 20-50° C., preferably ethanolic or methanolic NaOH or KOH.

Yet again, when the basic reaction has ended, if the product is to be a substituted 1,2,3,4-tetrahydroquinoline-2-carboxylic acid derivative of formula I where R⁴=C(O)NR¹¹R¹², the reaction product can be reacted with propanone-2-semicarbazone in water/glacial acetic acid at 30-60° C.

Likewise, when the basic reaction has ended, if the product is to be a substituted 1,2,3,4-tetrahydroquinoline-2-carboxylic acid derivative of formula I where R⁴=C(S)NR¹¹R¹², the reaction product can be reacted with CS₂ and a hydrazine in water/NaOH at 30-60° C.

As the final possibility to be mentioned here, when the basic reaction has ended, if the product is to be a substituted 1,2,3,4-tetrahydroquinoline-2-carboxylic acid derivative of formula I where R⁴=alkyl, benzyl or phenethyl, the reaction product can be reacted with an appropriate alkylating halide, benzyl halide or phenethyl halide and a suitable base, preferably sodium hydride or potassium tert-butylate, in a solvent, for example ethanol, at between 0 and 100° C. (J. Org. Chem., 1947, 12, 760; Zh. Obshch. Khim., 1942, 12, 418).

Under many of said reaction conditions, OH, SH and NH₂ groups may possibly enter into unwanted secondary reactions. It is therefore preferable to provide them with protecting groups or, in the case of NH₂, to replace it with NO₂, and to cleave the protecting group, or reduce the NO₂ group, before purifying the end product. The present invention therefore also provides a modification of the process described above wherein, in the starting compounds, at least one OH group has been replaced by an OSi(Ph)₂tert-butyl group, at least one SH group has been replaced by an S-p-methoxybenzyl group and/or at least one NH₂ group has been replaced by an NO₂ group, and at least one and preferably all of the OSi(Ph)₂tert-butyl groups are cleaved with tetrabutylammonium fluoride in tetrahydrofuran, and/or at least one and preferably all of the p-methoxybenzyl groups are cleaved with a metal amine, preferably sodamine, and/or at least one and preferably all of the NO₂ groups are reduced to NH₂ before the end product is purified.

Furthermore, carboxylic acid or thiocarboxylic acid groups are sometimes unstable under said reaction conditions, so it is preferable to use their methyl esters in the reactions and then to saponify the product of the process with KOH solution or NaOH solution in methanol at 40° C.-60° C. The invention therefore also provides a modification of the processes described above wherein, before the end product is purified, a product of the process having at least one C(O)OCH₃, OC(O)OCH₃ and/or C(S)OCH₃ group is saponified with KOH solution or NaOH solution in methanol or ethanol at 0° C.-100° C., preferably at 40° C.-60° C.

Therefore, it can also be favorable, for the preparation of (salts according to the invention of) substituted 1,2,3,4-tetrahydroquinoline-2-carboxylic acid derivatives of formula I in which R³=H, to use for the basic process starting materials of formula III in which R³% H and is preferably alkyl, especially CH₃ and C₂H₅. After the basic process and also the consecutive reactions that may follow it, the reaction product is saponified with an appropriate base, preferably NaOH (for example 6N) or KOH, in ethanol or methanol at temperatures of between 0° C. and 100° C., preferably of 40° C.-60° C. (Organikum, 1990, p. 418).

The following procedure is used to prepare the salts, especially the physiologically acceptable salts with cations or bases:

One equivalent of a compound of formula I, preferably an imino acid or a carboxylic acid, particularly where R³=H, is suspended in a small volume of water and one equivalent of 1 N aqueous alkaline solution, for example NaOH or KOH, is added. If the solubility is poor, methanol is added dropwise until the compound has completely dissolved. After stirring at room temperature, the solution is concentrated on a rotary evaporator and the residual solution is frozen at low temperatures in an isopropanol dry ice mixture and freeze-dried. The salts, especially of imino acids or carboxylic acids and preferably the sodium or potassium salts, are usually obtained as colorless solids.

An alternative possibility is to prepare the potassium or sodium salts with potassium or sodium trimethylsilanolate (E. D. Laganis, B. L. Chenard, Tetrahedron Letters, 25, 5831-5834 (1984)). Potassium or sodium trimethylsilanolate is dissolved under nitrogen in an organic solvent (e.g. dichloromethane, toluene or THF) and the ester or acid is added all at once. The reaction mixture is stirred for several hours at room temperature and filtered. The usually colorless solid is washed and dried under vacuum. The potassium or sodium salts are obtained as solids.

The salts according to the invention of substituted 1,2,3,4-tetrahydroquinoline-2-carboxylic acid derivatives are toxicologically safe, so they are suitable as pharmaceutical active ingredients in drugs.

The invention therefore also provides a drug containing, as the active ingredient, at least one salt according to the invention of a substituted 1,2,3,4-tetrahydroquinoline-2-carboxylic acid derivative of formula I in the form shown, or in the form of the acid or base, or in the form of its salts, especially the physiologically acceptable salts, or in the form of its solvates, especially the hydrates, particularly in the form of its physiologically acceptable salts with cations or bases or with anions or acids, optionally in the form of its racemates or its pure stereoisomers, especially enantiomers or diastereoisomers, or in the form of mixtures of the stereoisomers, especially the enantiomers or diastereoisomers, in any desired mixing ratio, and optionally containing suitable additives and/or auxiliary substances and/or optionally other active ingredients.

The drugs according to the invention can be administered as liquid dosage forms, i.e. injection solutions, drops or juices, or as semisolid dosage forms, i.e. granules, tablets, pellets, patches, capsules, plasters or aerosols, and optionally contain, apart from at least one salt according to the invention of a substituted tetrahydroquinoline derivative, excipients, fillers, solvents, diluents, colorants and/or binders, depending on the galenical form. The choice of auxiliary substances and the amounts thereof to be used depend on whether the drug is to be administered orally, perorally, parenterally, intravenously, intraperitoneally, intradermally, intramuscularly, intranasally, buccally, rectally or locally, for example to infections on the skin, mucous membranes or eyes. Suitable preparations for oral administration are those in the form of tablets, dragees, capsules, granules, drops, juices and syrups, and suitable preparations for parenteral, topical and inhalational administration are solutions, suspensions, readily reconstitutable dry preparations and sprays. Salts according to the invention of substituted tetrahydroquinoline derivatives in a depot in dissolved form or in a plaster, optionally with the addition of skin penetration promoters, are suitable preparations for transdermal application. Preparative forms for oral or transdermal administration can release the salts according to the invention of substituted tetrahydroquinoline derivatives in a delayed manner. The amount of active ingredient to be administered to the patient varies according to the patient's weight, the mode of administration, the indication and the severity of the disease. Conventionally, 2 to 500 mg/kg of at least one salt according to the invention of a substituted tetrahydroquinoline derivative of formula I are administered.

Preferably, the salts according to the invention of substituted 1,2,3,4-tetrahydroquinoline-2-carboxylic acid derivatives are used for the treatment of pain, especially chronic and neuropathic pain, as well as migraine, so the invention also provides the use of at least one salt according to the invention of a substituted 1,2,3,4-tetrahydroquinoline-2-carboxylic acid derivative of formula I, optionally in the form of its racemates or its pure stereoisomers, especially enantiomers or diastereoisomers, or in the form of mixtures of the stereoisomers, especially the enantiomers or diastereoisomers, in any desired mixing ratio, for the preparation of a drug for the treatment of pain, especially neuropathic and/or chronic pain, and/or for the treatment of migraine.

The affinity for the NMDA receptor gives rise to other areas of application since it is known that NMDA antagonists have inter alia a neuroprotective action and can therefore also be used satisfactorily for syndromes associated with neurodegeneration and neural damage, such as Parkinson's disease and Huntington's chorea, etc. Other indications of the NMDA antagonists according to the invention are epilepsy, glaucoma, osteoporosis, ototoxicity, withdrawal symptoms associated with alcohol and/or drug abuse, stroke, including related cerebral ischemia, cerebral infarction, cerebral edema, hypoxia and anoxia, and also use for anxiolysis and in anesthesia. The invention therefore also provides the use of at least one salt according to the invention of a substituted 1,2,3,4-tetrahydroquinoline-2-carboxylic acid derivative of formula I, optionally in the form of its racemates or its pure stereoisomers, especially enantiomers or diastereoisomers, or in the form of mixtures of the stereoisomers, especially the enantiomers or diastereoisomers, in any desired mixing ratio, for the preparation of a drug for the treatment/prophylaxis of epilepsy, Parkinson's disease, Huntington's chorea, glaucoma, ototoxicity, withdrawal symptoms associated with alcohol and/or drug abuse, stroke, cerebral ischemia, cerebral infarction, cerebral edema, hypoxia and anoxia, and/or for anxiolysis and/or anesthesia.

Surprisingly, it has been found that the substituted 1,2,3,4-tetrahydroquinoline-2-carboxylic acid derivatives according to the invention are also very suitable for other indications and especially for the treatment of urinary incontinence, pruritus, tinnitus aurium and/or diarrhea. The present invention therefore also contemplates the use of at least one salt according to the invention of a substituted 1,2,3,4-tetrahydroquinoline-2-carboxylic acid derivative of formula I, optionally in the form of its racemates or its pure stereoisomers, especially enantiomers or diastereoisomers, or in the form of mixtures of the stereoisomers, especially the enantiomers or diastereoisomers, in any desired mixing ratio, for the preparation of a drug for the treatment of urinary incontinence, pruritus, tinnitus aurium and/or diarrhea.

However, the compounds according to the invention are also effective in other indications. The present invention therefore also contemplates the use of at least one salt according to the invention of a substituted 1,2,3,4-tetrahydroquinoline-2-carboxylic acid derivative of formula I, optionally in the form of its racemates or its pure stereoisomers, especially enantiomers or diastereoisomers, or in the form of mixtures of the stereoisomers, especially the enantiomers or diastereoisomers, in any desired mixing ratio, for the treatment/prophylaxis of schizophrenia, Alzheimer's disease, psychosis due to a raised amino acid level, AIDS-related dementia, encephalomyelitis, Tourette's syndrome, perinatal asphyxia, inflammatory and allergic reactions, depression, drug and/or alcohol abuse, gastritis, diabetes, cardiovascular disease, respiratory tract disease, cough and/or mental disease.

The invention also provides a method of treating a non-human mammal or a human in need of a treatment for medically relevant symptoms, by the administration of a therapeutically effective dose of a salt according to the invention of a substituted 1,2,3,4-tetrahydroquinoline-2-carboxylic acid derivative of formula I, optionally in the form of its racemates or its pure stereoisomers, especially enantiomers or diastereoisomers, or in the form of mixtures of the stereoisomers, especially the enantiomers or diastereoisomers, in any desired mixing ratio, or a drug according to the invention. The invention relates especially to appropriate methods for the treatment of pain, especially neuropathic and/or chronic pain, and/or for the treatment of migraine, for the treatment of urinary incontinence, pruritus, tinnitus aurium and/or diarrhea, for the treatment/prophylaxis of epilepsy, Parkinson's disease, Huntington's chorea, glaucoma, osteoporosis, ototoxicity, withdrawal symptoms associated with alcohol and/or drug abuse, stroke, cerebral ischemia, cerebral infarction, cerebral edema, hypoxia and anoxia, and/or for anxiolysis and/or anesthesia, or for the treatment/prophylaxis of schizophrenia, Alzheimer's disease, psychosis due to a raised amino acid level, AIDS-related dementia, encephalomyelitis, Tourette's syndrome, perinatal asphyxia, inflammatory and allergic reactions, depression, drug and/or alcohol abuse, gastritis, diabetes, cardiovascular disease, respiratory tract disease, cough and/or mental disease.

The invention is further illustrated below by means of Examples, which are provided for purposes of illustration and are not intended to, nor should be deemed to be, limiting.

### EXAMPLES

The Examples which follow show compounds according to the invention, their preparation and efficacy tests performed therewith.

The following information is generally applicable:

The chemicals and solvents used were acquired commercially from the conventional suppliers (Acros, Avocado, Aldrich, Fluka, Lancaster, Maybridge, Merck, Sigma, TCI, etc.) or synthesized.

In particular, some of the compounds used are synthesized as synthetic moieties by known procedures before the basic synthesis described below.

The thin layer chromatography tests were carried out with HPTLC precoated plates, silica gel 60 F 254, from E. Merck, Darmstadt.

The yields of the compounds prepared are not optimized.

The analysis was performed by ESI mass spectroscopy.

The compounds are numbered, the data in brackets corresponding in principle to the number of the assigned compound.

### Example 0

Basic Process for the Preparation of the Basic Compounds of Formula I

a) One equivalent of aniline derivative and one equivalent of trifluoroacetic acid are dissolved in 6 ml/mmol of acetonitrile at room temperature, with stirring, and 1.1 equivalents of ethyl glyoxalate (50% in toluene) or 1.1 equivalents of glyoxalic acid monohydrate are then added. After ten minutes, 3 equivalents of the olefin component are added and the course of the reaction is followed by thin layer chromatography (solvent system: diethyl ether/hexane, 1:1). The reaction has ended after 2 hours (TLC check). An excess of saturated aqueous sodium hydrogencarbonate solution is added to the reaction mixture and the organic phase is extracted three times with diethyl ether. The organic phase is washed with water until the washings are neutral and dried over magnesium sulfate, the magnesium sulfate is filtered and washed with diethyl ether, the product phase is concentrated and the product is isolated by recrystallization or silica gel chromatography.

The 1,2,3,4-tetrahydroquinoline-2-carboxylic acid ester is characterized by ESI mass spectrometry.

b) Optional subsequent preparation of the free 1,2,3,4-tetrahydroquinoline-2-carboxylic acids

The above-described 1,2,3,4-tetrahydroquinoline-2-carboxylic acid ester (1 equivalent) is dissolved in 4 ml/mmol of ethanol, and 1.2 equivalents of 6 N aqueous sodium hydroxide solution are added at room temperature, with stirring. The course of the ester saponification is followed by thin layer chromatography (solvent system: diethyl ether/hexane, 1:1) and has ended after 30 minutes (TLC check). The reaction mixture is concentrated on a rotary evaporator, taken up in approximately 10 ml of water and adjusted to pH 1 with 32% HCl. The aqueous solution is extracted five times with diethyl ether, dried over magnesium sulfate and concentrated.

Automated Process

A round-bottom screw-threaded glass tube (diameter 16 mm, length 125 mm) was provided with a stirrer and sealed with a screw cap comprising a septum. The tube was placed in a stirring block thermostatted at 20° C. The following reactants were then pipetted successively into the tube:

1 ml of a solution of 0.1 M trifluoroacetic acid and 0.1 M aniline component in acetonitrile;

1 ml of a 0.11 M solution of the aldehyde in acetonitrile;

1 ml of a 0.3 M solution of the olefin in acetonitrile.

The reaction mixture was stirred for 10 h at 20° C. in one of the stirring blocks. The reaction solution was then filtered, the tube being rinsed with twice 1.5 ml of 7.5% NaHCO₃ solution.

2 ml of ethyl acetate were added to the reaction mixture on a vortex shaker and the mixture was shaken. It was centrifuged briefly to form a phase boundary. This boundary was detected optically and the organic phase was pipetted off. Next, 2 ml of ethyl acetate were added to the aqueous phase, the mixture was shaken and centrifuged and the organic phase was pipetted off. The combined organic phases were dried over 2.4 g of MgSO₄ (granulated). The solvent was stripped in a vacuum centrifuge.

The free 1,2,3,4-tetrahydroquinoline-2-carboxylic acid or the ester is characterized by ESI mass spectrometry.

In principle, in the case of compounds where R³≠H, both the automated process and the normal basic process can be followed by a saponification using methods known to those skilled in the art, for example with KOH solution or NaOH solution in methanol or ethanol at 0° C.-100° C., preferably at 40° C.-60° C.

Examples 1 to 58, which now follow, show the preparation of the basic compounds of formula I by one of the processes of Example 0, salts according to the invention of said compounds being prepared after the particular preparation described.

### Example 1

7,9-Dichloro-3a,4,5,9b-tetrahydro-3H-cyclopenta [c]quinoline-4-carboxylic acid (1)

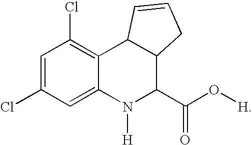

Compound 1 was saponified with 1.0 ml of sodium hydroxide solution (6 N, water) in 20.0 ml of ethanol. The ethanolic solution was concentrated on a rotary evaporator, the residue was taken up in water, 6 N HCl was added and the aqueous solution was extracted three times with ether. The organic phase was washed with water until the washings were neutral, dried over magnesium sulfate and concentrated on a rotary evaporator.

An ESI-MS was run to characterize the product:

MS (EI) m/z: 284 (M*)

### Example 2

8-Chloro-3a,4,5,9b-tetrahydro-3H-cyclopenta [c]quinoline-4-carboxylic acid (2)

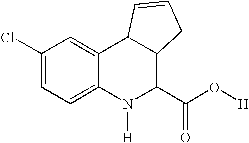

Compound 2 was saponified with 1.0 ml of sodium hydroxide solution (6 N, water) in 20.0 ml of ethanol. The ethanolic solution was concentrated on a rotary evaporator, the residue was taken up in water, 6 N HCl was added and the aqueous solution was extracted three times with ether. The organic phase was washed with water until the washings were neutral, dried over magnesium sulfate and concentrated on a rotary evaporator.

An ESI-MS was run to characterize the product:

MS (EI) m/z: 250 (M*)

### Example 3

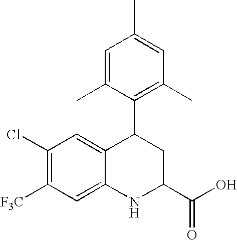

6-Chloro-7-trifluoromethyl-4-(2,4,6-trimethylphenyl)-1,2,3,4-tetrahydroquinoline-2-carboxylic acid (3)

Compound 3 was prepared by the basic process from 5.0 mmol of 4-chloro-4-trifluoromethylaniline, 5.5 mmol of glyoxalic acid monohydrate and 15.0 mmol of 2,4,6-trimethylstyrene in 30 ml of acetonitrile.

An ESI-MS was run to characterize the product:

MS (EI) m/z: 398.1 (M*)

### Example 4

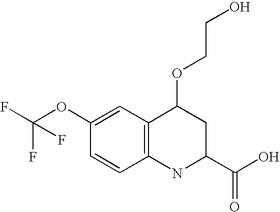

4-(2-Hydroxyethoxy)-6-trifluoromethoxy-1,2,3,4-tetrahydroquinoline-2-carboxylic acid (4)

Compound 4 was prepared by the automated process from 4-(trifluoromethoxy)aniline, glyoxylic acid and ethylene glycol monovinyl ether.

### Example 5

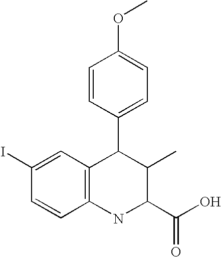

6-Iodo-4-(4-methoxyphenyl)-3-methyl-1,2,3,4-tetrahydroquinoline-2-carboxylic acid (5)

Compound 5 was prepared by the automated process from 4-iodoaniline, glyoxylic acid and trans-anethole.

### Example 6

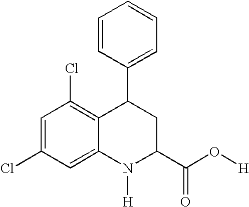

5,7-Dichloro-4-phenyl-1,2,3,4-tetrahydroquinoline-2-carboxylic acid (6)

Compound 6 was prepared by the basic process from 5.0 mmol of 3,5-dichloroaniline, 5.5 mmol of ethyl glyoxalate solution (50%, toluene), 15.0 mmol of styrene and 5.0 mmol of trifluoroacetic acid in 30.0 ml of acetonitrile. The subsequent saponification was carried out with 1.0 ml of sodium hydroxide solution (6 N, water) in 20.0 ml of ethanol.

An ESI-MS was run to characterize the product:

MS (EI) m/z: (M*) 315

### Example 7

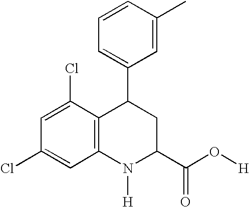

5,7-Dichloro-4-m-tolyl-1,2,3,4-tetrahydroquinoline-2-carboxylic acid (7)

Compound 7 was prepared by the basic process from 5.0 mmol of 3,5-dichloroaniline, 5.5 mmol of ethyl glyoxalate solution (50%, toluene), 15.0 mmol of 3-methylstyrene and 5.0 mmol of trifluoroacetic acid in 30.0 ml of acetonitrile. The subsequent saponification was carried out with 1.0 ml of sodium hydroxide solution (6 N, water) in 20.0 ml of ethanol.

An ESI-MS was run to characterize the product:

MS (EI) m/z: (M*) 335

### Example 8

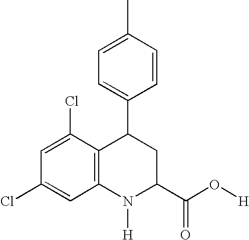

5,7-Dichloro-4-p-tolyl-1,2,3,4-tetrahydroquinoline-2-carboxylic acid (8)

Compound 8 was prepared by the basic process from 5.0 mmol of 3,5-dichloroaniline, 5.5 mmol of ethyl glyoxalate solution (50%, toluene), 15.0 mmol of 4-methylstyrene and 5.0 mmol of trifluoroacetic acid in 30.0 ml of acetonitrile. The subsequent saponification was carried out with 1.0 ml of sodium hydroxide solution (6 N, water) in 20.0 ml of ethanol.

An ESI-MS was run to characterize the product:

MS (EI) m/z: (M*) 335

### Example 9

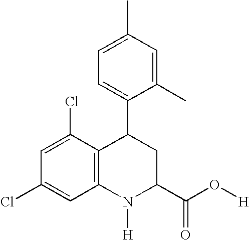

5,7-Dichloro-4-(2,4-dimethylphenyl)-1,2,3,4-tetrahydroquinoline-2-carboxylic acid (9)

Compound 9 was prepared by the basic process from 5.0 mmol of 3,5-dichloroaniline, 5.5 mmol of ethyl glyoxalate solution (50%, toluene), 15.0 mmol of 2,4-dimethylstyrene and 5.0 mmol of trifluoroacetic acid in 30.0 ml of acetonitrile. The subsequent saponification was carried out with 1.0 ml of sodium hydroxide solution (6 N, water) in 20.0 ml of ethanol.

An ESI-MS was run to characterize the product:

MS (EI) m/z: (M*) 349

### Example 10

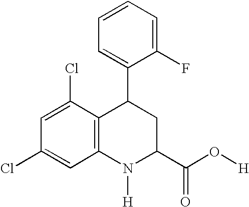

5,7-Dichloro-4-(2-fluorophenyl)-1,2,3,4-tetrahydroquinoline-2-carboxylic acid (10)

Compound 10 was prepared by the basic process from 5.0 mmol of 3,5-dichloroaniline, 5.5 mmol of ethyl glyoxalate solution (50%, toluene), 15.0 mmol of 2-fluorostyrene and 5.0 mmol of trifluoroacetic acid in 30.0 ml of acetonitrile. The subsequent saponification was carried out with 1.0 ml of sodium hydroxide solution (6 N, water) in 20.0 ml of ethanol.

An ESI-MS was run to characterize the product:

MS (EI) m/z: (M*) 339

### Example 11

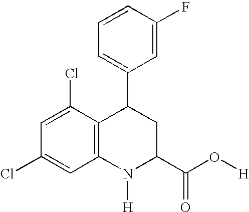

5,7-Dichloro-4-(3-fluorophenyl)-1,2,3,4-tetrahydroquinoline-2-carboxylic acid (11)

Compound 11 was prepared by the basic process from 5.0 mmol of 3,5-dichloroaniline, 5.5 mmol of ethyl glyoxalate solution (50%, toluene), 15.0 mmol of 3-fluorostyrene and 5.0 mmol of trifluoroacetic acid in 30.0 ml of acetonitrile. The subsequent saponification was carried out with 1.0 ml of sodium hydroxide solution (6 N, water) in 20.0 ml of ethanol.

An ESI-MS was run to characterize the product:

MS (EI) m/z: (M*) 340

### Example 12

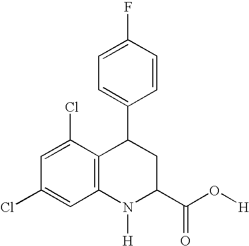

5,7-Dichloro-4-(4-fluorophenyl)-1,2,3,4-tetrahydroquinoline-2-carboxylic acid (12)

Compound 12 was prepared by the basic process from 5.0 mmol of 3,5-dichloroaniline, 5.5 mmol of ethyl glyoxalate solution (50%, toluene), 15.0 mmol of 4-fluorostyrene and 5.0 mmol of trifluoroacetic acid in 30.0 ml of acetonitrile. The subsequent saponification was carried out with 1.0 ml of sodium hydroxide solution (6 N, water) in 20.0 ml of ethanol.

An ESI-MS was run to characterize the product:

MS (EI) m/z: (M*) 340

### Example 13

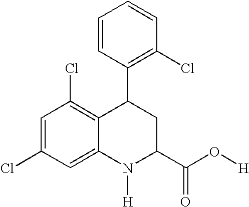

5,7-Dichloro-4-(2-chlorophenyl)-1,2,3,4-tetrahydroquinoline-2-carboxylic acid (13)

Compound 13 was prepared by the basic process from 5.0 mmol of 3,5-dichloroaniline, 5.5 mmol of ethyl glyoxalate solution (50%, toluene), 15.0 mmol of 2-chlorostyrene and 5.0 mmol of trifluoroacetic acid in 30.0 ml of acetonitrile. The subsequent saponification was carried out with 1.0 ml of sodium hydroxide solution (6 N, water) in 20.0 ml of ethanol.

An ESI-MS was run to characterize the product:

MS (EI) m/z: (M*) 356

### Example 14

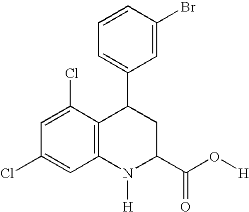

4-(3-Bromophenyl)-5,7-dichloro-1,2,3,4-tetrahydroquinoline-2-carboxylic acid (14)

Compound 14 was prepared by the basic process from 5.0 mmol of 3,5-dichloroaniline, 5.5 mmol of ethyl glyoxalate solution (50%, toluene), 15.0 mmol of 3-bromostyrene and 5.0 mmol of trifluoroacetic acid in 30.0 ml of acetonitrile. The subsequent saponification was carried out with 1.0 ml of sodium hydroxide solution (6 N, water) in 20.0 ml of ethanol.

An ESI-MS was run to characterize the product:

MS (EI) m/z: (M*) 401

### Example 15

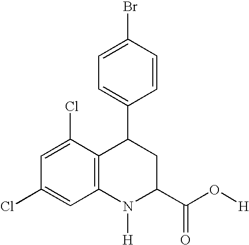

4-(4-Bromophenyl)-5,7-dichloro-1,2,3,4-tetrahydroquinoline-2-carboxylic acid (15)

Compound 15 was prepared by the basic process from 5.0 mmol of 3,5-dichloroaniline, 5.5 mmol of ethyl glyoxalate solution (50%, toluene), 15.0 mmol of 4-bromostyrene and 5.0 mmol of trifluoroacetic acid in 30.0 ml of acetonitrile. The subsequent saponification was carried out with 1.0 ml of sodium hydroxide solution (6 N, water) in 20.0 ml of ethanol.

An ESI-MS was run to characterize the product:

MS (EI) m/z: (M*) 401

### Example 16

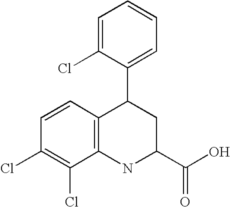

7,8-Dichloro-4-(2-chlorophenyl)-1,2,3,4-tetrahydroquinoline-2-carboxylic acid (16)

Compound 16 was prepared by the automated process from 2,3-dichloroaniline, glyoxylic acid and 2-chlorostyrene.

### Example 17

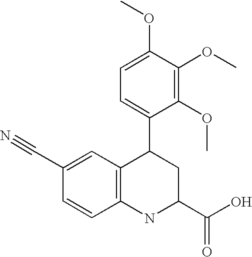

6-Cyano-4-(2,3,4-trimethoxyphenyl)-1,2,3,4-tetrahydroquinoline-2-carboxylic acid (17)

Compound 17 was prepared by the automated process from 4-aminobenzonitrile, glyoxylic acid and 2,3,4-tetramethoxystyrene.

### Example 18

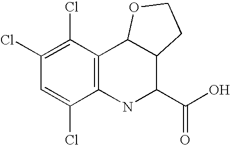

6,8,9-Trichloro-2,3,3a,4,5,9b-hexahydrofuro [3,2-c]quinoline-4-carboxylic acid (18)

Compound 67 was prepared by the automated process from 2,4,5-trichloroaniline, glyoxylic acid and 2,3-dihydrofuran.

### Example 19

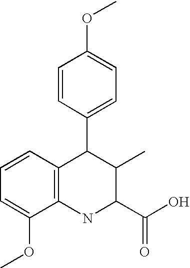

8-Methoxy-4-(4-methoxyphenyl)-3-methyl-1,2,3,4-tetrahydroquinoline-2-carboxylic acid (19)

Compound 19 was prepared by the automated process from 2-methoxyaniline, glyoxylic acid and trans-anethole.

### Example 20

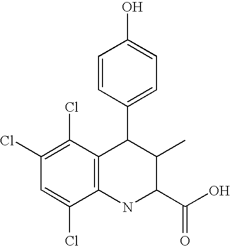

5,6,8-Trichloro-4-(4-hydroxyphenyl)-3-methyl-1,2,3,4-tetrahydroquinoline-2-carboxylic acid (20)

Compound 20 was prepared by the automated process from 2,3,5-trichloroaniline, glyoxylic acid and 2-propenylphenol.

### Example 21

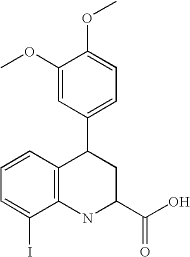

4-(3,4-Dimethoxyphenyl)-8-iodo-1,2,3,4-tetrahydroquinoline-2-carboxylic acid (21)

Compound 21 was prepared by the automated process from 2-iodoaniline, glyoxylic acid and 3,4-dimethoxystyrene.

### Example 22

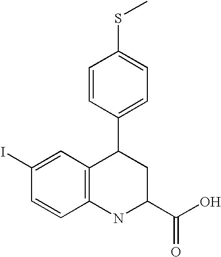

6-Iodo-4-(4-methylsulfanylphenyl)-1,2,3,4-tetrahydroquinoline-2-carboxylic acid (22)

Compound 22 was prepared by the automated process from 4-iodoaniline, glyoxylic acid and 1-methylsulfanyl-4-vinylbenzene.

### Example 23

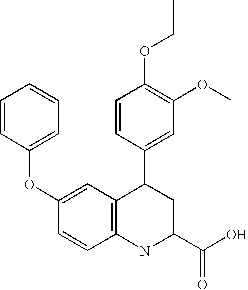

4-(4-Ethoxy-3-methoxyphenyl)-6-phenoxy-1,2,3,4-tetrahydroquinoline-2-carboxylic acid (23)

Compound 23 was prepared by the automated process from 4-phenoxyaniline, glyoxylic acid and 1-ethoxy-2-methoxy-4-vinylbenzene.

### Example 24

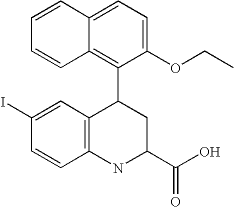

4-(2-Ethoxynaphthalen-1-yl)-6-iodo-1,2,3,4-tetrahydroquinoline-2-carboxylic acid (24)

Compound 24 was prepared by the automated process from 4-iodoaniline, glyoxylic acid and 2-ethoxy-1-vinylnaphthalene.

### Example 25

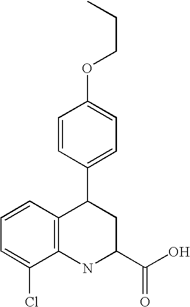

8-Chloro-4-(4-propoxyphenyl)-1,2,3,4-tetrahydroquinoline-2-carboxylic acid (25)

Compound 25 was prepared by the automated process from 2-chloroaniline, glyoxylic acid and 4-propoxystyrene.

### Example 26

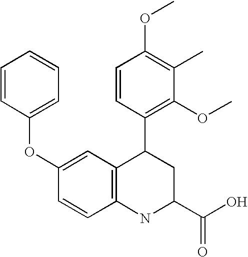

4-(2,4-Dimethoxy-3-methylphenyl)-6-phenoxy-1,2,3,4-tetrahydroquinoline-2-carboxylic acid (26)

Compound 26 was prepared by the automated process from 4-phenoxyaniline, glyoxylic acid and 2,4-dimethoxy-3-methylstyrene. Example 27 to 102 were prepared analogously.

| Example | Name |
|---|---|
| 27 | 4-anthracen-9-yl-6-chloro-8-methyl-1,2,3,4-tetrahydroquinoline-2- |
| | carboxylic acid |
| 28 | 6-sec-butyl-4-naphthalen-1-yl-1,2,3,4-tetrahydroquinoline-2- |
| | carboxylic acid |
| 29 | 4-(4-hydroxyphenyl)-3-methyl-8-phenoxy-1,2,3,4- |
| | tetrahydroquinoline-2-carboxylic acid |
| 30 | 8-chloro-6-fluoro-4-naphthalen-2-yl-1,2,3,4-tetrahydroquinoline-2- |
| | carboxylic acid |
| 31 | 4-(4-methoxyphenyl)-3-methyl-6-phenoxy-1,2,3,4- |
| | tetrahydroquinoline-2-carboxylic acid |
| 32 | 6-chloro-8-fluoro-4-m-tolyl-1,2,3,4-tetrahydroquinoline-2-carboxylic acid |
| 33 | 8-chloro-6-fluoro-4-m-tolyl-1,2,3,4-tetrahydroquinoline-2-carboxylic acid |
| 34 | 4-(4-bromophenyl)-6-chloro-8-fluoro-1,2,3,4-tetrahydroquinoline-2-carboxylic acid |
| 35 | 7,8-dichloro-4-(2,4-dimethylphenyl)-1,2,3,4-tetrahydroquinoline-2-carboxylic acid |
| 36 | 6-chloro-4-(4-chlorophenyl)-7-trifluoromethyl-1,2,3,4- |
| | tetrahydroquinoline-2-carboxylic acid |
| 37 | 4-(2-chlorophenyl)-6-cyano-1,2,3,4-tetrahydroquinoline-2-carboxylic acid |
| 38 | 6-bromo-8-chloro-4-(2,4-dimethylphenyl)-1,2,3,4- |
| | tetrahydroquinoline-2-carboxylic acid |
| 39 | 6-bromo-4-(2-bromophenyl)-8-chloro-1,2,3,4-tetrahydroquinoline-2-carboxylic acid |
| 40 | 4-(4-hydroxy-3-methoxyphenyl)-3-methyl-6-methylsulfanyl-1,2,3,4- |
| | tetrahydroquinoline-2-carboxylic acid |
| 41 | 6-cyano-3,4-bis(4-methoxyphenyl)-1,2,3,4-tetrahydroquinoline-2-carboxylic acid |
| 42 | ethyl 8-chloro-6-fluoro-3,4-bis(4-methoxyphenyl)-1,2,3,4- |
| | tetrahydroquinoline-2-carboxylate |
| 43 | 5,7-dichloro-4-(4-chlorophenyl)-1,2,3,4-tetrahydroquinoline-2-carboxylic acid |
| 44 | 5,7-dichloro-4-(3-chlorophenyl)-1,2,3,4-tetrahydroquinoline-2-carboxylic acid |
| 45 | 5,7-dichloro-4-(4-chlorophenyl)-1,2,3,4-tetrahydroquinoline-2- |
| 46 | 1,3-dichloro-5,6,6a,7,8,12b-hexahydrobenzo[k]phenanthridine-6-carboxylic acid |
| 47 | 1,3-dichloro-5,6a,7,11b-tetrahydro-6H-indeno[2,1-c]quinoline-6-carboxylic acid |
| 48 | 5,7-dichloro-4-(3,5-dimethylphenyl)-1,2,3,4-tetrahydroquinoline-2-carboxylic acid |
| 49 | 7,9-dichloro-2,3,3a,4,5,9b-hexahydro-1H-cyclopenta[c]quinoline-4-carboxylic acid |

### Example 50

Receptor binding (glycine binding site of the NMDA receptor channel)

The tests for determining the affinity of the compounds of formula I according to the invention for the glycine binding site of the NMDA receptor channel were carried out on brain membrane homogenates (homogenate of the cortex and hippocampus areas of the brain of male rats, Wistar strain) [B. M. Baron, B. W. Siegel, B. L. Harrison, R. S. Gross, C. Hawes and P. Towers, Journal Of Pharmacology and Experimental Therapeutics, vol. 279, p. 62, 1996].

For this purpose, the cortex and hippocampus were freed without cutting from freshly removed rat brains, homogenized in 5 mmol/l TRIS acetate buffer, 0.32 mol/l sucrose, pH 7.4 (10 ml/g of fresh weight), using a Potter homogenizer (Braun/Melsungen, 10 piston strokes at 500 rpm), with ice cooling, and then centrifuged for 10 minutes at 1000 g and 4° C. The first supernatant was collected and the sediment was homogenized again with 5 mmol/l TRIS acetate buffer, 0.32 mol/l sucrose, pH 7.4 (5 ml/g of original fresh weight), using the Potter homogenizer (10 piston strokes at 500 rpm), with ice cooling, and centrifuged for 10 minutes at 1000 g and 4° C. The resulting supernatant was combined with the supernatant from the first centrifugation and centrifuged at 17,000 g for 20 minutes at 4° C. The supernatant after this centrifugation was discarded and the membrane sediment was taken up with 5 mmol/l TRIS acetate buffer, pH 8.0 (20 ml/g of original fresh weight), and homogenized with 10 piston strokes at 500 rpm.

The membrane homogenate was then incubated for 1 hour at 4° C. and centrifuged for 30 minutes at 50,000 g and 4° C. The supernatant was discarded and the centrifuge tube containing the membrane sediment was sealed with Parafilm and frozen for 24 hours at −20° C. On the following day the membrane sediment was thawed, taken up with ice-cold 5 mmol/l TRIS acetate buffer, 0.1% saponin (w/v), pH 7.0 (10 ml/g of original fresh weight), homogenized with 10 piston strokes at 500 rpm and then centrifuged for 20 minutes at 50,000 g and 4° C. The resulting supernatant was discarded and the sediment was taken up in a small volume with 5 mmol/l TRIS acetate buffer, pH 7.0 (approximately 2 ml/g of original fresh weight), and homogenized again with 10 piston strokes at 500 rpm. After determination of the protein content, the membrane homogenate was adjusted to a protein concentration of 10 mg protein/ml with 5 mmol/l TRIS acetate buffer, pH 7.0, and frozen in aliquots until tested.

For the receptor binding test, aliquots were thawed, diluted to 1:10 with 5 mmol/l TRIS acetate buffer, pH 7.0, homogenized with 10 piston strokes at 500 rpm using the Potter homogenizer (10 piston strokes at 500 rpm), with ice cooling, and centrifuged for 60 minutes at 55,000 g and 4° C. The supernatant was decanted and the membrane sediment was adjusted to a protein concentration of 1 mg/ml with ice-cold 50 mmol/I TRIS acetate buffer, pH 7.0, homogenized again with 10 piston strokes at 500 rpm and kept in suspension by stirring on a magnetic stirrer in an ice bath. 100 μl aliquots of this membrane homogenate were used per ml of preparation in the receptor binding test (0.1 mg protein/ml in the final preparation).

In the binding test, 50 mmol/l TRIS acetate buffer, pH 7.0, was used as the buffer and 1 nmol/l (³H)-MDL 105.519 (B. M. Baron et al., 1996) was used as the radioactive ligand. The proportion of non-specific binding was determined in the presence of 1 mmol/l glycine.

In other preparations the compounds according to the invention were added in concentration series and the displacement of the radioactive ligand from its specific binding to the glycine binding site of the NMDA receptor channel was measured. The preparations, in triplicate in each case, were incubated for 120 minutes at 4° C. and then harvested by means of filtration through glass fiber filter mats (GF/B) in order to determine the radioactive ligand bound to the membrane homogenate. The radioactivity retained on the glass fiber filters was measured in a β counter after the addition of scintillator.

The affinity of the compounds according to the invention for the glycine binding site of the NMDA receptor channel was calculated as the IC₅₀ value (concentration causing 50% displacement of the radioactive ligand from its specific binding) according to the law of mass action by means of non-linear regression and is indicated in Table 1 as the Ki value (mean of 3 independent experiments) after conversion (according to the Cheng-Prussoff relationship), or as the percentage of the previously bound radioactive ligand (see above) which is displaced from its specific binding by a concentration of 10 μmol/l of the substance according to the invention to be tested.

TABLE 1

Glycine
binding site of the
NMDA receptor channel

| | | Displacement |
|---|---|---|
| Example | Ki(μmol/l) | (%, 10 μmol/l) |
| 2 | 0.3 | 100 |

### Example 51

NMDA/Glycine-Induced Ionic Fluxes on RNA-Injected Xenopus Oocytes

The test to determine functional changes of the NMDA receptor channel due to the compound of formula I according to the invention was performed on oocytes of the South African clawed frog, Xenopus laevis. This was done by forming neuronal NMDA receptor channels in oocytes after the injection of rat brain RNA and measuring ionic fluxes triggered by the co-application of NMDA and glycine.

Stage V and VI Xenopus oocytes (Dumont, J. N., Journal of Morphology, vol. 136, 1972) were microinjected with total RNA from adult rat brain tissue (100-130 ng/cell) and kept at 20° C. for up to 10 days in culture medium (composition in mmol/l: 88.0 NaCl, 1.0 KCl, 1.5 CaCl₂, 0.8 MgSO₄, 2.4 NaHCO₃, 5 HEPES, 100 IU/ml penicillin, 100 μg/ml streptomycin, pH 7.4). Transmembrane ionic fluxes were recorded by the conventional two-electrode voltage clamp technique at a holding potential of −70 mV (P. Bloms-Funke, P. M. Madeja, U. Musshoff, E.-J. Speckmann, Neuroscience Letters, vol. 205, p. 115, 1996). The data were plotted and the experimental apparatus controlled using the OTC interface and Cellworks software (npi, FRG). The compounds according to the invention were added to a nominally Mg²⁺-free medium (composition in mmol/l: 89.0 NaCl, 1.0 KCl, 1.8 CaCl₂, 2.4 NaHCO₃, 5 HEPES, pH 7.4) and applied systemically by means of a concentration clamp (npi, FRG). To test substance effects mediated via the glycine B binding site of the NMDA receptor channel, the glycine dose-effect curve was plotted with and without the particular compound according to the invention. To do this, NMDA in a fixed concentration of 100 μmol/I was cumulatively co-applied with glycine in increasing concentrations (0-100 μmol/l). The experiment was then repeated in the same manner with a fixed concentration of the compound according to the invention. To estimate the selectivity for NMDA versus AMPA receptor channels, the effects of the compound according to the invention (10 μmol/l) was additionally studied on ionic fluxes triggered by AMPA (100 μmol/l). The current amplitudes were normalized to that of the control response to the co-application of NMDA (100 μmol/l) with glycine (10 μmol/l). The data were analyzed with Igor-Pro software (Version 3.1, WaveMetrics, USA). All the results were expressed as the mean±standard error (SEM) of at least 3 experiments on different oocytes from at least two frogs. The significance for unpaired and paired measurable variables is determined by the Mann-Whitney U-test and the Wilcoxon test (Sysstat, SPSS Inc., USA) respectively. EC₅₀ values are calculated according to the following formula:

Y=Yₘᵢₙ+(Yₘₐₓ−Yₘᵢₙ)/(1+(X/EC₅₀)^{-P})

(Yₘᵢₙ=minimum test value, Yₘₐₓ=maximum test value, Y=relative current amplitude, X=concentration of test substance, p=slope factor). On right shifting of the glycine dose-effect curve, the pA₂ value of the compound according to the invention was determined graphically by means of Schild regression. Concentration ratios were calculated using the EC₅₀ values, which were determined independently for each dose-effect curve.

The right shifting of the glycine dose-effect curve is shown for Example no. 1 (relative amplitude: current amplitude, normalized to the response after application of NMDA/glycine (100/10 μmol/l)). The results for selected compounds according to the invention in respect of their effects on the glycine dose-effect curve and on AMPA-induced ionic fluxes have been collated in Table 2.

TABLE 2
Effects of the compounds according to the invention on ionic
fluxes triggered by NMDA/glycine and AMPA on RNA-
injected oocytes

| | | AMPA-induced ionic |
|---|---|---|
| | | fluxes |
| | NMDA/glycine-induced | Inhibition at 10 μmol/l |
| | ionic fluxes | of the compounds |
| | pA₂ value relating to the | according to the |
| Example no. | glycine dose-effect curve | invention |
| 1 | 6.40 | 5.4% (n = 2) |

### Example 52

Formalin Test on the Rat

The experiments for determining the antinociceptive action of the compounds of formula I according to the invention were performed in the formalin test on male rats (Sprague-Dawley, 150-170 g).

In the formalin test a distinction is made between the first (early) phase (0-15 min after the formalin injection) and the second (late) phase (15-60 min after the formalin injection) (D. Dubuisson, S. G. Dennis, Pain, 4, 161-174 (1977)). The early phase represents a model for acute pain as a direct reaction to the formalin injection, while the late phase is regarded as a model for persistent (chronic) pain (T. J. Coderre, J. Katz, A. L. Vaccarino, R. Melzack, Pain, vol. 52, p. 259, 1993).

The compounds according to the invention were studied in the second phase of the formalin test in order to make predictions about the actions of substances on chronic/inflammatory pain.

A single subcutaneous formalin injection (50 μl, 5%) in the dorsal side of the right back paw of free-moving experimental animals induced a nociceptive reaction which is represented by the following behavioral parameters: lifting and holding of the affected paw (score 1), shaking or twitching (score 2), licking and biting (score 3). The differing modes of behavior triggered by the formalin injection were recorded continuously by observing the animals in the late phase of the formalin test, and differently weighted in an evaluation. Normal behavior, where the animal distributes its weight evenly over all four paws, was recorded as a score of 0. The time of administration before the formalin injection was chosen according to the mode of administration of the compounds according to the invention (intraperitoneal: 15 min, intravenous: 5 min). After the injection of substances that have an antinociceptive action in the formalin test, the described modes of behavior (score 1-3) of the animals are reduced or even eliminated. The comparison was made with control animals which had received vehicle (solvent) before the administration of formalin. The nociceptive behavior was calculated as the so-called pain rate (PR). The different behavioral parameters were differently weighted (factor 0, 1, 2, 3). The calculation was performed in subintervals of 3 min according to the following formula:

PR=[(T₀×0)+(T₁×1)+(T₂×2)+(T₃×3)]/180

T₀, T₁, T₂ and T₃ corresponding to the time in seconds in which the animal exhibited the mode of behavior 0, 1, 2 or 3, respectively. The number of animals in the substance and vehicle groups, n, is 10 in each case. Based on the PR calculations, the substance effect was determined as a percentage change relative to the control. The ED₅₀ calculations were performed by regression analysis.

All the compounds according to the invention which were tested showed a moderately strong to strong inhibition of the formalin-induced nociception.

The results of selected studies in the formalin test on the rat are collated in the Table below.

TABLE 3

| | | | % inhibition of the |
|---|---|---|---|
| | Mode of | | formalin-induced |
| Compound | administration | Dose [mg/kg] | nociception |
| 1 | i.p. | 21.5 | 64.5 |

### Example 53

### General Preparation of the Salts According to the Invention from the Compounds of One of Examples 1-49

One equivalent of the compound of one of Examples 1 to 49, preferably an imino acid or carboxylic acid, is suspended in a small volume of water and one equivalent of 1 N aqueous alkaline solution, preferably NaOH or KOH, is added. If the solubility is poor, methanol is added dropwise until the compound has completely dissolved. After stirring for 30 minutes at room temperature, the solution is concentrated on a rotary evaporator and the residual solution is frozen at −60° C. in an isopropanol/dry ice mixture and freeze-dried. The salts, especially of imino acids and preferably the sodium or potassium salts, are usually obtained as colorless solids.

An alternative possibility is to prepare the potassium or sodium salts with potassium or sodium trimethylsilanolate (E. D. Laganis, B. L. Chenard, Tetrahedron Letters, 25, 5831-5834 (1984)). Potassium or sodium trimethylsilanolate is dissolved under nitrogen in an organic solvent (dichloromethane, toluene, THF) and the ester or acid is added all at once. The reaction mixture is stirred for four hours at room temperature and filtered. The usually colorless solid is washed with diethyl ether and dried under vacuum. The potassium or sodium salts are obtained as solids.

### Example 54

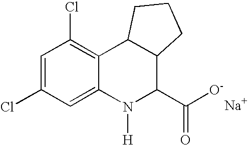

7,9-Dichloro-2,3,3a,4,5,9b-hexahydro-1H-cyclopenta [c]quinoline-4-carboxylate; sodium salt (54)

7,9-Dichloro-2,3,3a,4,5,9b-hexahydro-1H-cyclopenta[c]quinoline-4-carboxylic acid (49) is treated according to Example 106 to give 7,9-dichloro-2,3,3a,4,5,9b-hexahydro-1H-cyclopenta [c]quinoline-4-carboxylate; sodium salt (54).

### Example 55

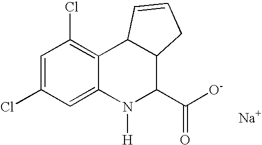

7,9-Dichloro-3a,4,5,9b-tetrahydro-3H-cyclopenta [c]quinoline-4-carboxylate; sodium salt (55)

Compound 1 prepared according to Example 1 is treated according to Example 53 to give 7,9-dichloro-3a,4,5,9b-tetrahydro-3H-cyclopenta [c]quinoline-4-carboxylate; sodium salt (55).

Molecular weight calculated by ESI-MS: 284.14 g/mol; measured molecular weight: 282.3 (M−H), 238.4 (M-CO₂).

¹H NMR (d₆-DMSO/TMSₑₓₜ): d=2.15-2.40 ppm (m, 2H, CH₂); 3.35 ppm (q, 1H, CH); 3.50 ppm (m, 1H, CH); 4.05 ppm (dd, 1H, CH); 5.60 ppm (m, 1H, olefin H); 5.70 ppm (m, 1H, NH); 5.80 ppm (M, 1H, olefin CH); 6.60 ppm (d, 1H, aryl CH); 6.85 ppm (d, 1H, aryl CH).

### Example 56

### Receptor Binding of the Salts According to the Invention at the Glycine Binding Site of the NMDA Receptor Channel

Compounds 54 and 55 are studied for receptor binding as explained in Example 50:

TABLE 4

Glycine binding site of the NMDA receptor channel

| | | Displacement |
|---|---|---|
| Example | Ki(μmol/l) | (%, 10 μmol/l) |
| 54 | | 97 |
| 55 | 0.35 | 90 |

### Example 57

### Formalin Test

Compound 55 was studied in the formalin test as described in Example 52.

TABLE 5

| | | | % inhibition of the |
|---|---|---|---|
| | Mode of | | formalin-induced |
| Compound | administration | Dose [mg/kg] | nociception |
| 55 | i.v. | 68.1 | 56 |

### Example 58

### Parenteral Mode of Administration

38.5 g of compound 55 are dissolved in 11 of water for injection at room temperature and then adjusted to isotonic conditions by the addition of anhydrous glucose for injection.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A substituted 1,2,3,4-tetrahydroquinoline-2-carboxylic acid compound corresponding to formula I: 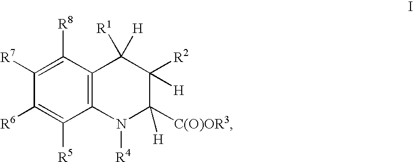
wherein
R¹ and R² together form the following, each of which is monosubstituted or polysubstituted or unsubstituted:
—(CH₂)ₙ—, where n=3-10
—CH═CH—CH₂—, —CH₂—CH═CH—,
—CH═CH—CH₂—CH₂—, —CH₂—CH₂—CH═CH—,
—CH₂—CH═CH—CH₂—,
—CH₂—CH═CH—CH₂—CH₂—, —CH₂—CH₂—CH═CH—CH₂—,
—CH₂—CH₂—CH═CH—CH₂—CH₂—,
—O—CH₂—CH₂—, —CH₂—CH₂—O—,
—O—CH₂—CH₂—CH₂—, —CH₂—CH₂—CH₂—O—,
—CH₂—O—CH₂—,
—CH₂—CH₂—O—CH₂—, —CH₂—O—CH₂—CH₂—, 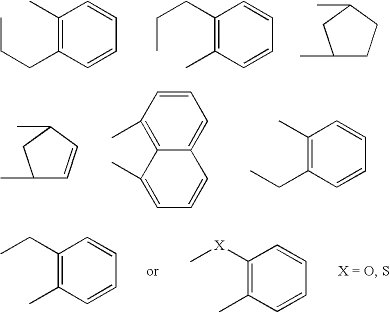
R³ represents
H; C₁-C₁₈-alkyl, C₂-C₁₈-alkenyl or C₂-C₁₈-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by N, S or O; alkylaryl or alkylheteroaryl, each of which is monosubstituted or polysubstituted or unsubstituted; and aryl or heteroaryl, each of which is monosubstituted or polysubstituted or unsubstituted;
R⁴ represents
R^{4a} or ZR^{4a}, where Z=C₁-C₆-alkyl, C₂-C₆-alkenyl or C₂-C₆-alkynyl, each of which is branched or unbranched and monosubstituted or polysubstituted or unsubstituted; and R^{4a} represents
H; C₁-C₁₂-alkyl, C₂-C₁₂-alkenyl or C₂-C₁₂-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; and aryl or heteroaryl, each of which is monosubstituted or polysubstituted or unsubstituted;
C(O)R⁹, C(O)OR⁹, C(S)R⁹, C(S)OR⁹ or S(O₂)R⁹, where R⁹ represents
H; C₁-C₁₀-alkyl, C₂-C₁₀-alkenyl or C₂-C₁₀-alkynyl, each of which is branched or unbranched and monosubstituted or polysubstituted or unsubstituted; C3-C₈-cycloalkyl which is saturated or unsaturated and monosubstituted or polysubstituted or unsubstituted, or a corresponding heterocycle in which at least one ring C atom is replaced by S, O or N; alkylaryl or alkylheteroaryl, each of which is monosubstituted or polysubstituted or unsubstituted; and aryl or heteroaryl, each of which is monosubstituted or polysubstituted or unsubstituted, especially phenethyl, 1-adamantyl, 2-adamantyl, 1-naphthyl or 2-naphthyl, 2-, 3- or 4-pyridyl or thiazolyl;
SR¹⁰, where R¹⁰ represents aryl or heteroaryl, each of which is monosubstituted or polysubstituted or unsubstituted;
C(O)NR¹¹R¹², C(O)NR¹¹NR¹²R¹³, C(NR¹¹)NR¹²R¹³, C(S)NR¹¹R¹² or C(S)NR¹¹NR¹²R¹³, where R¹¹, R¹² and R¹³ independently represent
H; C₁-C₁₈-alkyl, C₂-C₁₈-alkenyl or C₂-C₁₈-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; alkylaryl or alkylheteroaryl, each of which is monosubstituted or polysubstituted or unsubstituted; and aryl or heteroaryl, each of which is monosubstituted or polysubstituted or unsubstituted;
R⁵, R⁶, R⁷ and R⁸ independently represent
H; F; Cl; Br; I; CN; NO₂; and C₁-C₁₀-alkyl, C₂-C₁₀-alkenyl or C₂-C₁₀-alkynyl, each of which is branched or unbranched and monosubstituted or polysubstituted or unsubstituted;
OR¹⁴, OC(O)R¹⁴, OC(S)R¹⁴, C(O)R¹⁴, C(O)OR¹⁴, C(S)R¹⁴, C(S)OR¹⁴, SR¹⁴, S(O)R¹⁴ or S(O₂)R¹⁴, where R¹⁴ represents
H; C₁-C₁₀-alkyl, C₂-C₁₀-alkenyl or C₂-C₁₀-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; alkylaryl or alkylheteroaryl, each of which is monosubstituted or polysubstituted or unsubstituted; and aryl or heteroaryl, each of which is monosubstituted or polysubstituted or unsubstituted;
NR¹⁵R¹⁶, NR¹⁵C(O)R¹⁶, C(NR¹⁵)NR¹⁶R¹⁷, NR¹⁵C(S)R¹⁶, C(S)NR¹⁵R¹⁶, C(S)NR¹⁵NR¹⁶R¹⁷ or S(O₂)NR¹⁵R¹⁶, where R¹⁵, R¹⁶ and R¹⁷ independently represent
H; O; C₁-C₁₈-alkyl, C₂-C₁₈-alkenyl or C₂-C₁₈-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; alkylaryl or alkylheteroaryl, each of which is monosubstituted or polysubstituted or unsubstituted; and aryl or heteroaryl, each of which is monosubstituted or polysubstituted or unsubstituted; or
R¹⁵ and R¹⁶ or R¹⁶ and R¹⁷ together form a C₃-C₈-cycloalkyl which is saturated or unsaturated and monosubstituted or polysubstituted or unsubstituted, or a corresponding heterocycle in which at least one ring C atom is replaced by S, O or N; and
R⁵ and R⁶, R⁶ and R⁷ or R⁷ and R⁸ together form
═CR¹⁸—CH═CH—CH═ or ═CH—CR¹⁸═CH—CH═, where R¹⁸ represents
H; F; Cl; Br; I; OH; and C₁-C₁₀-alkyl, C₂-C₁₀-alkenyl or C₂-C₁₀-alkynyl, each of which is branched or unbranched and monosubstituted or polysubstituted or unsubstituted,
in the form of a salt thereof with a physiologically acceptable acid or in the form of a salt thereof with a base,
provided that
if R¹ and R² together form —CH═CH—CH₂— or 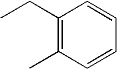
and R³ is (−)-p-menthan-3-ol, R⁷≠Cl and R⁵, R⁶ and R⁸≠H simultaneously,
if R¹ and R² together form —CH═CH—CH₂— and R³ is CH₃, R⁷≠H, Cl or OCH₃ and R⁵, R⁶ and R⁸≠H simultaneously,
if R^{1b} and R^{2a} together form —CH═CH—CH₂— and R³ is H, R⁷≠OCH₃ or C(O)NH₂ and R⁵, R⁶ and R⁸≠H, R⁵ and R⁷ ≠CH₃ and R⁶ and R⁸# H, or R⁵≠OCH₃ and R⁶, R⁷ and R⁸≠H simultaneously, or
if R^{1b} and R^{2a} together form 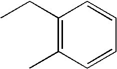
or —O—CH₂—CH₂— and R³ is C₂H₅, R⁷≠H, Cl, CH₃, OCH₃ or NO₂ and R⁵, R⁶ and R⁸≠H, or R⁵ ≠NO₂ and R⁶, R⁷ and R⁸≠H simultaneously.

2. The compound of claim 1, wherein if R¹ and R² together form —CH═CH—CH₂— or 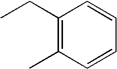
and R³ is menthol or borneol, R⁷≠Cl and R⁵, R⁶ and R⁸≠H simultaneously.

3. The compound of claim 1, wherein said compound is present in the form of a pure enantiomer.

4. The compound of claim 1, wherein said compound is present in the form of a pure diastereoisomer.

5. The compound of claim 1, wherein said compound is present in the form of a mixture of stereoisomers.

6. The compound of claim 1, wherein said compound is present in the form of a racemic mixture.

7. The compound of claim 1, wherein said compound is present in the form of an NH₄+, monopotassium, dipotassium, magnesium or calcium salt.

8. The compound of claim 1, wherein said compound is present in the form of an NH₄+ salt.

9. The compound of claim 1, wherein R⁴ represents
H; C₁-C₁₀-alkyl, C₂-C₁₀-alkenyl or C₂-C₁₀-alkynyl, each of which is branched or unbranched and monosubstituted or polysubstituted or unsubstituted; and C₃-C₈-cycloalkyl which is saturated or unsaturated and monosubstituted or polysubstituted or unsubstituted; and
C(O)R⁹, where R⁹ represents H; C₁-C₁₀-alkyl, C₂-C₁₀-alkenyl or C₂-C₁₀-alkynyl, each of which is branched or unbranched and monosubstituted or polysubstituted or unsubstituted; C₃-C₈-cycloalkyl which is saturated or unsaturated and monosubstituted or polysubstituted or unsubstituted; and aryl or heteroaryl, each of which is monosubstituted or polysubstituted or unsubstituted,

10. The compound of claim 1, wherein R⁴ represents
C(O)R⁹, where R⁹ represents phenethyl, 1-adamantyl, 2-adamantyl, 1-naphthyl or 2-naphthyl, 2-, 3- or 4-pyridyl or thiazolyl.

11. The compound of claim 1, wherein R⁴ represents
H, CH₃ or C₂H₅.

12. The compound of claim 1, wherein R³ represents
H; C₁-C₁₀-alkyl, C₂-C₁₀-alkenyl or C₂-C₁₀-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by N or O;
alkylaryl which is monosubstituted or polysubstituted or unsubstituted;
and aryl or heteroaryl, each of which is monosubstituted or polysubstituted or unsubstituted.

13. The compound of claim 1, wherein R³ represents
H; C₁-C₄-alkyl which is branched or unbranched and monosubstituted or polysubstituted or unsubstituted; and phenyl, benzyl or phenethyl which is monosubstituted or polysubstituted or unsubstituted.

14. The compound of claim 1, wherein R³ represents
H, CH₃ or C₂H₅.

15. The compound of claim 1, wherein R¹ and R² together form
—O—CH₂—CH₂—, (—CH₂—)ₙ where n=3-6, —CH═CH—CH₂—, —CH═CH—CH₂—CH₂—,

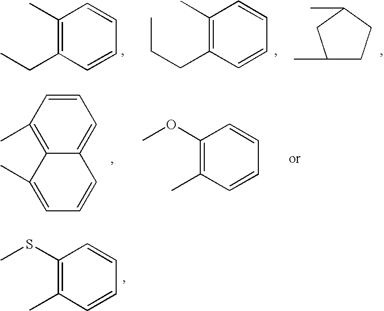

16. The compound of claim 1, wherein R¹ and R² together form
(—CH₂—)ₙ where n=preferably 3 or 6, —CH═CH—CH₂— or —CH═CH—CH₂—CH₂—.

17. The compound of claim 1, wherein R⁵, R⁶, R⁷ and R⁸ independently represent
H; F; Cl; Br; I; CN; NO₂; and C₁-C₁₀-alkyl, C₂-C₁₀-alkenyl or C₂-C₁₀-alkynyl, each of which is branched or unbranched and monosubstituted or polysubstituted or unsubstituted;
OR¹⁴, C(O)R¹⁴, C(O)OR¹⁴ or SR¹⁴; and
NR¹⁵R¹⁶ or NR¹⁵C(O)R¹⁶, R¹⁵ and R¹⁶ independently represent
H; O; C₁-C₁₀-alkyl, C₂-C₁₀-alkenyl or C₂-C₁₀-alkynyl, each of which is branched or unbranched and monosubstituted or polysubstituted or unsubstituted.

18. The compound of claim 1, wherein R⁵, R⁶, R⁷ and R⁸ independently represent
H; F; Cl; Br; I; CN; NO₂; and C₁-C₆-alkyl, C₂-C₆-alkenyl or C₂-C₆-alkynyl, each of which is branched or unbranched and monosubstituted or polysubstituted or unsubstituted;
OR¹⁴, C(O)R¹⁴, C(O)OR¹⁴ or SR¹⁴, where R¹⁴ represents
H; C₁-C₄-alkyl which is branched or unbranched and monosubstituted or polysubstituted or unsubstituted; and aryl which is monosubstituted or polysubstituted or unsubstituted.

19. The compound of claim 1, wherein R⁵, R⁶, R⁷ and R⁸ independently represent
H; F; Cl; Br; I; CN; and C₁-C₄-alkyl which is branched or unbranched and monosubstituted or polysubstituted or unsubstituted;
OR¹⁴ or SR¹⁴, where R¹⁴ represents
C₁-C₄-alkyl which is branched or unbranched and monosubstituted or polysubstituted or unsubstituted; and aryl which is monosubstituted or polysubstituted or unsubstituted.

20. The compound of claim 1, wherein R⁵, R⁶, R⁷ and R⁸ independently represent
H; F; Cl; Br; I; CN; CH₃; CF₃; t-butyl; i-butyl; —OCH₃; —OCF₃; —SCH₃ or —O-phenyl.

21. The compound of claim 1, wherein R⁵, R⁶, R⁷ and R⁸ independently represent
H; F; Cl; Br; I; CN; NO₂; CF₃; and C₁-C₆-alkyl, C₂-C₆-alkenyl or C₂-C₆-alkynyl, each of which is branched or unbranched and unsubstituted;
OR¹⁴, C(O)R¹⁴, C(O)OR¹⁴ or SR¹⁴, where R¹⁴ represents
H; C₁-C₄-alkyl which is branched or unbranched and monosubstituted or polysubstituted or unsubstituted; and aryl which is monosubstituted or polysubstituted or unsubstituted.

22. The compound of claim 1, wherein R⁵, R⁶, R⁷ and R⁸ independently represent
H; F; Cl; Br; I; CN; CF₃; and C₁-C₄-alkyl which is branched or unbranched and unsubstituted;
OR¹⁴ or SR¹⁴, where R¹⁴ represents
C₁-C₄-alkyl which is branched or unbranched and monosubstituted or polysubstituted or unsubstituted; and aryl which is monosubstituted or polysubstituted or unsubstituted.

23. The compound of claim 1, wherein R⁵, R⁶, R⁷ and R⁸ independently represent
H; F; Cl; Br; I; CN; CH₃; CF₃; t-butyl; i-butyl; —OCH₃; —OCF₃; —SCH₃ or —O-phenyl.

24. The compound of claim 1, wherein
R⁵, R⁶ and R⁸ are H and R⁷ is Cl, or
R⁵ and R⁷ are H and R⁶ and R⁸ are Cl.

25. The compound of claim 1, wherein said compound is selected from the group consisting of the salts of:
7,9-dichloro-3a,4,5,9b-tetrahydro-3H-cyclopenta quinoline-4-carboxylic acid,
8-chloro-3a,4,5,9b-tetrahydro-3H-cyclopenta [c]quinoline-4-carboxylic acid,
6,8,9-trichloro-2,3,3a,4,5,9b-hexahydrofuro [3,2-c]quinoline-4-carboxylic acid,
1,3-dichloro-5,6,6a,7,8,12b-hexahydrobenzo [k]phenanthridine-6-carboxylic acid,
1,3-dichloro-5,6a,7,11b-tetrahydro-6H-indeno [2,1-c]quinoline-6-carboxylic acid and
7,9-dichloro-2,3,3a,4,5,9b-hexahydro-1H-cyclopenta [c]quinoline-4-carboxylic acid.

26. The compound of claim 1, wherein said compound is selected from the group consisting of the salts of:
sodium 7,9-dichloro-3a,4,5,9b-tetrahydro-3H-cyclopenta [c]quinoline-4-carboxylate or
sodium 7,9-dichloro-2,3,3a,4,5,9b-hexahydro-1H-cyclopenta [c]quinoline-4-carboxylate.

27. A process for producing a substituted 1,2,3,4-tetrahydroquinoline-2-carboxylic acid compound corresponding to formula I of claim 1, wherein R⁴=H, 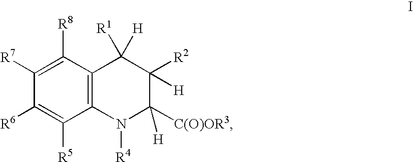
comprising the steps of:
reacting an aniline corresponding to formula II, a glyoxalic acid ester or a glyoxalic acid corresponding to formula III and an olefin of formula IV, with trifluoroacetic acid.

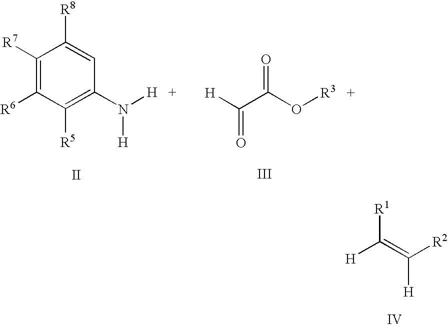

28. The process of claim 27, wherein said step of reacting is carried out at a temperature between 0° C. and 100° C.

29. The process of claim 27, wherein at least one of R¹, R² and R³ are independently provided with a protective group.

30. The process of claim 27, further comprising the step of saponifying any ester groups existing when the reacting step has ended or bringing the product formed when the reacting step has ended into contact with a strong base, which strong base may already contain the desired cation, in order to form a salt.

31. The process of claim 27, wherein the duration of the reaction is 0.25-12 hours.

32. The process of claim 27, wherein the duration of the reaction is no longer than 2 hours.

33. The process of claim 27, wherein the reaction is carried out at a temperature of between 20° C. and 40° C.

34. The process of claim 27, wherein the reaction is carried out at room temperature.

35. The process of claim 27, wherein the reaction is a single-vessel reaction.

36. A process for producing a substituted 1,2,3,4-tetrahydroquinoline-2-carboxylic acid compound corresponding to formula I of claim 1, wherein R⁴≠H, 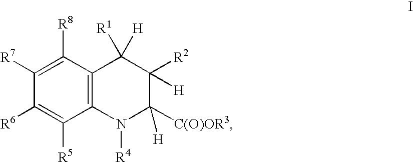
comprising the steps of:
reacting an aniline corresponding to formula II, a glyoxalic acid ester or a glyoxalic acid corresponding to formula III and an olefin of formula IV, with trifluoroacetic acid to form a reaction product wherein R⁴=H 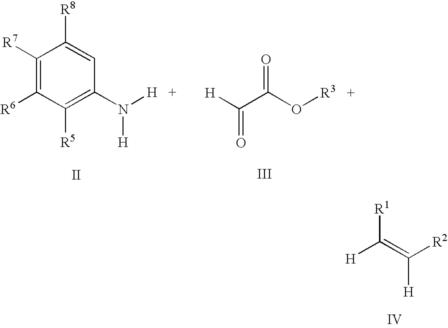
reacting said reaction product to substitute the H on R⁴ with
R^{4a} or ZR^{4a}, where Z=C₁-C₆-alkyl, C₂-C₆-alkenyl or C₂-C₆-alkynyl, each of which is branched or unbranched and monosubstituted or polysubstituted or unsubstituted; and R^{4a} represents
C₁-C₁₂-alkyl, C₂-C₁₂-alkenyl or C₂-C₁₂-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; and aryl or heteroaryl, each of which is monosubstituted or polysubstituted or unsubstituted;
C(O)R⁹, C(O)OR⁹, C(S)R⁹, C(S)OR⁹ or S(O₂)R⁹, where R⁹ represents
H; C₁-C₁₀-alkyl, C₂-C₁₀-alkenyl or C₂-C₁₀-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; alkylaryl or alkylheteroaryl, each of which is monosubstituted or polysubstituted or unsubstituted; and
aryl or heteroaryl, each of which is monosubstituted or polysubstituted or unsubstituted, especially phenethyl, 1-adamantyl, 2-adamantyl, 1-naphthyl or 2-naphthyl, 2-, 3- or 4-pyridyl or thiazolyl;
SR¹⁰, where R¹⁰ represents aryl or heteroaryl, each of which is monosubstituted or polysubstituted or unsubstituted;
C(O)NR¹¹R¹², C(O)NR¹¹NR¹²R¹³, C(NR¹¹)NR¹²R¹³, C(S)NR¹¹R² or C(S)NR¹¹NR¹²R¹³, where R¹¹, R¹² and R¹³ independently represent
H; C₁-C₁₈-alkyl, C₂-C₁₈-alkenyl or C₂-C₁₈-alkynyl, each of which is branched or unbranched and monosubstituted or polysubstituted or unsubstituted; C₃-C₈-cycloalkyl which is saturated or unsaturated and monosubstituted or polysubstituted or unsubstituted, or a corresponding heterocycle in which at least one ring C atom is replaced by S, O or N; alkylaryl or alkylheteroaryl, each of which is monosubstituted or polysubstituted or unsubstituted; and
aryl or heteroaryl, each of which is monosubstituted or polysubstituted or unsubstituted.

37. The process of claim 25, wherein in at least one of the aniline corresponding to formula II, the glyoxalic acid ester or glyoxalic acid compound corresponding to formula III or the benzofuran corresponding to formula IV, are independently provided with a protective group, said protective group being selected from the group consisting of
OSi(Ph)₂tert-butyl to replace an OH group;
S-p-methoxybenzyl to replace an SH group and
NO₂ to replace an NH₂ group and
before a purification step,
at least one OSi(Ph)₂tert-butyl group is cleaved with tetrabutylammonium fluoride in tetrahydrofuran;
at least one p-methoxybenzyl group is cleaved with a metal amide or
at least one NO₂ group is reduced to NH₂.

38. The process of claim 37, wherein said metal amide is sodium amide.

39. The process of claim 37, wherein, before a purification step,
all OSi(Ph)₂tert-butyl groups are cleaved with tetrabutylammonium fluoride in tetrahydrofuran;
all p-methoxybenzyl groups are cleaved with a metal amide or
all NO₂ groups are reduced to NH₂.

40. The process of claim 25, wherein a product of the process with at least one C(O)OCH₃ or C(S)OCH₃ group, or a product of the process wherein R³=C₁₋₄-alkyl, is saponified with KOH solution or NaOH solution in methanol or ethanol at a temperature of from 0° C.-100° C.

41. The process of claim 40, wherein said temperature is from 40° C.-60° C.

42. The process of claim 40, wherein in said product of the process, R³=CH₃ or C₂H₅.

43. A pharmaceutical composition, comprising:
at least one salt of a substituted 1,2,3,4-tetrahydroquinoline-2-carboxylic acid compound corresponding to formula I of claim 1 and
an auxiliary agent.

44. The pharmaceutical composition of claim 43, wherein said compound is present in the form of a pure enantiomer or pure diastereoisomer.

45. The pharmaceutical composition of claim 43, wherein said compound is present in the form of a mixture of stereoisomers.

46. The pharmaceutical composition of claim 43, wherein said compound is present in the form of a racemic mixture.

47. A method of alleviating pain in a mammal, said method comprising administering to said mammal an effective pain alleviating amount of a compound according to claim 1.

48. The method of claim 47, wherein said pain is neuropathic or chronic pain.

49. The method of claim 47, wherein said pain is pain from a migraine.

50. A method of treating urinary incontinence, pruritus, tinnitus aurium or diarrhea in a mammal, said method comprising administering to said mammal an effective amount of a compound according to claim 1.

51. A method of treating or inhibiting epilepsy, Parkinson's disease, Huntington's chorea, glaucoma, osteoporosis, ototoxicity, the withdrawal symptoms associated with alcohol or drug abuse, stroke, cerebral ischaemia, cerebral infarcts, cerebral oedema, hypoxia, anoxia or for anxiolysis or anaesthesia in a mammal, said method comprising administering to said mammal an effective amount of a compound according to claim 1.

52. A method of treating or inhibiting schizophrenia, Alzheimer's disease, psychosis due to increased amino acid levels, AIDS dementia, encephalomyelitis, Tourette's syndrome, perinatal asphyxia, inflammatory and allergic reactions, depression, drug or alcohol abuse, gastritis, diabetes, cardiovascular diseases, respiratory diseases, coughing or mental illnesses, said method comprising administering to said mammal an effective amount of a compound according to claim 1.
